(19) United States
(12) Patent Application Publication
(10) Pub. No.: US 2022/0359076 A9
(43) Pub. Date: Nov. 10, 2022

(54) COVID-19 SCREENING SYSTEM, APPARATUS, METHOD, AND GRAPHICAL USER INTERFACE

(71) Applicants: Narasimheswara Sarma Velamuri, Houston, TX (US); Marcus Joel Christian Persson Rydberg, Houston, TX (US)

(72) Inventors: Narasimheswara Sarma Velamuri, Houston, TX (US); Marcus Joel Christian Persson Rydberg, Houston, TX (US)

(21) Appl. No.: 17/360,991

(22) Filed: Jun. 28, 2021

Related U.S. Application Data
(63) Continuation-in-part of application No. 16/773,804, filed on Jan. 27, 2020.
Application No. 16/773,804 is a division of application No. 14/998,182, filed on Dec. 24, 2015, now Pat. No. 10,592,637.
(60) Provisional application No. 63/044,878, filed on Jun. 26, 2020.
(60) Provisional application No. 62/096,754, filed on Dec. 24, 2014.
(60) Provisional application No. 62/116,701, filed on Feb. 16, 2015.

(51) Int. Cl. G16H 50/20; G16H 50/30; G16H 40/67

(52) U.S. Cl. CPC G16H 50/20; G16H 50/30; G16H 40/67

(57) ABSTRACT

Disclosed is a computer-implemented method and system for screening people for COVID-19. The method entails receiving data relating to a person in a computing system including a processor and memory coupled to the processor. The memory stores programmable instructions executable by the processor. The presence of COVID-19 is determined based on the data. The data is received using a graphical user interface.

This application claims the benefit of U.S. Provisional Application No. 63/044,878, filed on Jun. 26, 2020 (pending), and this application is also a Continuation-in-Part application of U.S. application Ser. No. 16/773,804, filed on Jan. 27, 2020 (pending), which is a divisional application of U.S. application Ser. No. 14/998,182, filed on Dec. 24, 2015 (now U.S. patent Ser. No. 10/592,637), which claims the benefit of U.S. Provisional Application No. 62/096,745, filed on Dec. 24, 2014 (now expired) and of U.S. Provisional Application No. 62/116,701 filed on Feb. 16, 2015 (now expired). The disclosures of each of the aforementioned applications is hereby incorporated by reference for all purposes and made a part of the present disclosure.

### FIELD

The present disclosure relates generally to a system, apparatus, method, and graphical user interface for screening, diagnosing, managing and\or treating a patient with a medical condition. In particular, the present disclosure is directed to screening or diagnosing a medical condition, and even more particularly, in respect to end organ damage due to infection. The present disclosure also relates to a computer-implemented method of screening a patient.

### BACKGROUND

Potentially life-threatening complications can arise from an immune response triggered by infection. Patients can develop a severe response to an infection from almost any medical condition including: surgery, minor medical or dental procedures, postpartum (i.e., complications from childbirth), trauma, animal bites, or infections acquired inside or outside the hospital. Chemicals released by the body to fight the infection trigger inflammatory responses. As sepsis progresses to septic shock, blood pressure drops dramatically, which may result in damage to multiple organ systems and even death. According to the CDC's National Center for Health Statistics, the number of people seen in U.S. hospitals in 2008 increased from about 621,000 in 2008 to over 1.1 million. See e.g., http://www.cdc.gov/sepsis/datareports/index.html. The number of cases has been rising each year.

Treatment of sepsis currently focuses on early recognition of the condition to minimize end-organ damage and dissemination of the infection. The hallmarks of sepsis are easily recognized at the bedside but initial evidence of sepsis are frequently missed by health care providers who are not looking for it. Also, due to the labor demands of today's health care enterprise, health care providers are simply unavailable to monitor important changes in patient status. By today's standard of care, timely treatment depends on the ability to diagnose sepsis early and entails the following: (i) intervention in the first three hours, including obtaining blood cultures to detect infection in the blood; (ii) early administration of broad-spectrum antibiotics; (iii) measurement of venous lactate as a marker of tissue hypoperfusion (a medical condition in which an organ or extremity doesn't have enough blood); and (iv) administration of intravenous crystalloids (fluids that contain electrolytes). Depending on information derived from the clinical situation, treatment may further entail: (i) initiation of central venous access (placement of an intravenous catheter in the patient's neck or groin to access large bore veins); (ii) measurement of arterial blood pressure by placement of a catheter in a patient's artery; and (iii) initiation of vasopressor medications (medications that raise blood pressure by causing very strong constriction of arteries).

Infectious diseases can spread rapidly and uncontrollably throughout populations. For example, the spread of a virus, such as a coronavirus (e.g., COVID-19), can cause a pandemic if the spread remains uncontrolled. The ability to quickly identify those that have and do not have an infectious disease can provide benefits to those seeking to reduce the effects and/or spread of the infectious disease.

### BRIEF SUMMARY

Disclosed herein are a system, method, apparatus, and graphical user interface for screening, managing, diagnosing and\or treating a patient with a medical condition. Also disclosed are such a system, method, apparatus, and graphical user interface for screening a patient for end organ damage due to infection. The system, method, apparatus, and graphical user interface are equally and more suitable for screening for sepsis, given that the associated complications and treatment are much the same. Disclosures described herein in relation to sepsis are typically applicable to end organ damage due to infection (and vice-versa). The method is preferably a computer-implemented method and the system preferably includes a handheld or locally stationed device connected with one or more processor(s), a memory storing programmable instructions, and input/output user interface.

In one aspect, a computer-implemented method of screening a patient for end organ damage due to infection is described. The method includes receiving patient data in a computing system including a processor and memory coupled to the processor, wherein the memory stores instructions executable by the processor. Such a computing system may be composed of a single hand-held computing device or an entire local or cloud-based network, or equal, and variations thereof and in between. The presence of Systematic Inflammatory Response Syndrome (SIRS) is determined and then, the presence or absence of a high probability of end organ damage due to infection is determined. Each of the determining procedure or step is performed by receiving, on or via a graphical user interface, one or more affirmative user selections presented on the graphical user interface. Preferably, the determining procedure or step includes executing instructions stored in memory, with said user selections as input, to determine patient status relating to the presence of Systematic Inflammatory Response Syndrome or the presence of a high probability of end organ damage due to infection. As described herein, software may be packaged in an Application, including computer programs with algorithms for determining or screening, as described above, with user or system supplied patient data as input.

In another aspect, a method of screening a patient for end organ damage due to infection is described. The method entails providing patient data relating to an identified patient into a computing system including a processor and memory coupled to the processor, wherein the memory stores programmable instructions executable by the processor, determining the presence of Systematic Inflammatory Response Syndrome, and determining the presence or absence of a high probability of end organ damage due to infection. Each of the preceding determining steps includes inputting, on a graphical user interface, one or more user selections presented on the graphical user interface and initiating execution of programmable instructions based on the inputted user selections.

In another aspect, a computing system is also described having at least one processor, at least one display, and memory coupled to the at least one processor. The memory stores programmable instructions executable by the processor to present a plurality of graphical user interfaces on the display, including a first graphical user interface and a second graphical user interface, and programmable instructions executable to determine the presence of a medical condition based on user selected patient information received on the graphical user interfaces. The first user interface presents, as user selection options, patient related observations or conditions (e.g., physical measurements), such that executing instructions includes comparing user selected patient related conditions received on the user interface with a screening criterion to indicate the presence of Systematic Inflammatory Response Syndrome. The second user interface presents, as user selection options, a plurality of clinical judgment-based parameters, such that executing instructions includes comparing user selected clinical judgment-based parameters received on the user interface with a screening criterion to indicate a high probability of end organ damage due to infection. The programmable instructions are further executable to generate the second user interface upon the indication of the presence of Systematic Inflammatory Response Syndrome. As further described herein, the user selections may be provided or prompted on graphical user interfaces of a hand-held computing device, such as tablet, and the user merely activates the prompt to make the selection. Execution of the programmable instructions (i.e., screening algorithm) may be initiated by activating a second, subsequent prompt.

Another embodiment includes computer-implemented method and system for screening a person for infection with coronavirus. Data from the person can be received into a memory of a computing system. The computing system can include a processor and the memory coupled to the processor. The memory includes programmable instructions executable by the processor stored thereon. The receiving includes, on a graphical user interface generated by the computing system, receiving one or more user selections or inputs. A risk level of a presence of coronavirus in the person can be determined by executing at least some of the programmable instructions based on the received data.

### BRIEF DESCRIPTION OF THE DRAWINGS

A more complete and thorough understanding of the present embodiments and advantages thereof may be acquired by referring to the following description taken in conjunction with the accompanying drawings:

FIG. 1 is a simplified block diagram illustrating a method of screening according to the present disclosure;

FIGS. 1A-1E is a flowchart illustrating a preferred method of screening, according to the present disclosure;

FIG. 2A is a simplified diagram illustrating an exemplary system for or through which one or more steps of the method of FIGS. 1, and 1A-1E may be performed, in accordance with an embodiment of the present disclosure;

FIGS. 2B and 2C are simplified schematics of an alternative exemplary system for or through which one or more step of the method of FIGS. 1 and 1A-1E may be performed, in accordance with an embodiment of the present disclosure;

FIGS. 3A-3C are representative graphical user interfaces for use with the system and method of FIG. 1, according to one embodiment of the present disclosure;

FIGS. 4A-4C are representative graphical user interfaces for use with the system and method of FIGS. 1 and 1A-1E, according to one embodiment of the present disclosure;

FIGS. 5A-5D are representative graphical user interfaces for use with, or which may be generated by, the system and method of FIGS. 1 and 1A-1E, according to one embodiment of the present disclosure;

FIG. 6 is a representative graphical user interface for displaying an acuity dashboard for use with, or which may be generated by, the system and method of FIGS. 1 and 1A-1E, according to one embodiment of the present disclosure;

FIG. 7 depicts an administrator dashboard of the GUI for tracking the occurrence of COVID-19;

FIG. 8 is the administrator dashboard of FIG. 8 with a status selection made;

FIG. 9 is an administrator dashboard of the GUI showing a history of COVID-19 screening;

FIG. 10 is the administrator dashboard of the GUI showing a history of COVID-19 screening with a particular day selected;

FIGS. 11A-11C depict an employee or patient dashboard of the GUI on a desktop computer, presenting screening questions to determine the risk of COVID-19;

FIG. 12 depicts an employee or patient dashboard of the GUI on a mobile device, presenting screening questions to determine the risk of COVID-19;

FIG. 13 is a screening results dashboard of the GUI, presenting the screening results and indicating a low or zero risk of COVID-19;

FIG. 14 is a screening results dashboard of the GUI, presenting the screening results and indicating a low or moderate risk of COVID-19;

FIG. 15 is a screening results dashboard of the GUI, presenting the screening results and indicating COVID-19 or a high risk of COVID-19;

FIG. 16 is pie chart of COVID-19 testing results;

FIG. 17 is a chart of factors related to COVID-19 spread;

FIG. 18 is chart showing the use of the COVID-19 screening system for handling employees based on their individual COVID-19 risk;

FIG. 19 is chart showing the use of the COVID-19 screening system for handling groups of employees in an organization;

FIG. 20 is a dashboard of the GUI for screening for COVID-19 showing COVID-19 risk geographically;

FIG. 21 is a chart showing the use of the COVID-19 screening system for assessing the organization's ecosystem;

FIG. 22 is a chart of benefits of using the COVID-19 screening system; and

FIG. 23 is a chart of benefits of using a screening system.

### DETAILED DESCRIPTION

Before any embodiments are explained in detail, it is to be understood that the systems, apparatus, methods, user interfaces, and products according to the present disclosure are not limited in its application to the details in the examples provided in this Detailed Description. Specific examples pertaining to the management and diagnosis of a patient, potentially with end organ damage due to infection or sepsis, are provided for illustration only. The arrangement of steps in the process or the components in the system described in respect to a patient with end organ damage due to infection or sepsis (potentially) may be varied in further embodiments in response to different conditions, objectives, and requirements. In such further embodiments, steps may be carried out in a manner involving different environmental conditions, analyses thereof, and responses thereto, as well as to different collections of data. Moreover, the description that follows includes exemplary apparatuses, methods, techniques, and instruction sequences that embody techniques of the disclosed subject matter. It is understood, however, that the described embodiments may be practiced without these specific details or employing only portions thereof.

As used herein, the terms “managing” or “screening” as directed to a patient or subject suspected of having a medical condition of interest may encompass one or more of screening, diagnosing, treating, and observing the patient or subject and collecting data therefrom. Moreover, the terms managing or screening encompasses such interaction with a patient or subject that does not result in or confirm the presence of the object medical condition (i.e., sepsis), as well as such interaction that does.

Notwithstanding the above, the present Detail Description relies on a generally computer-implemented and\or software-implemented process of screening a patient for end organ damage due to infection or sepsis to illustrate relevant concepts. The described process(es) also provides the preferred modes of carrying out the concepts in commercial applications. The present disclosure is, of course, not limited to screening, diagnosing, and managing for end organ damage due to infection or sepsis, as the concepts and embodiments provides a generalized platform for screening, diagnosing, and managing other medical conditions. Moreover, certain aspects of the described systems or processes, including certain graphical user interfaces and the use thereof, may be applicable to or in other patient care practices.

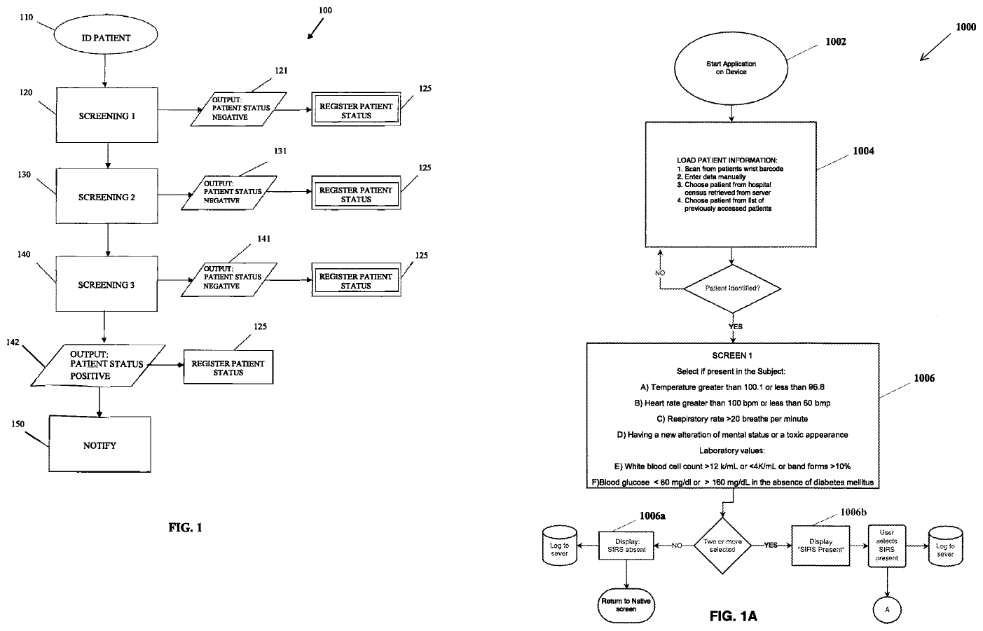

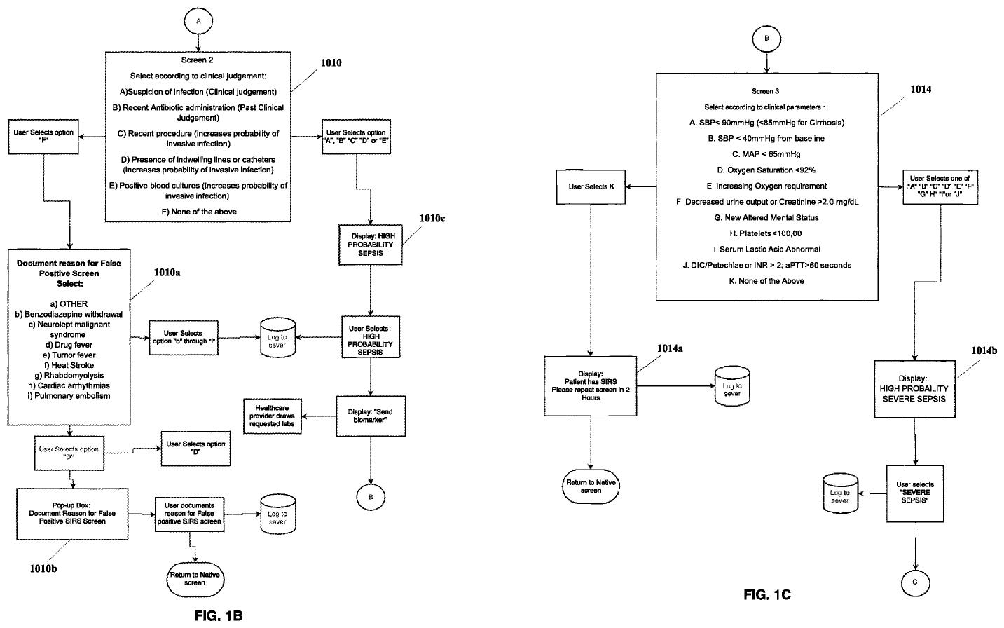

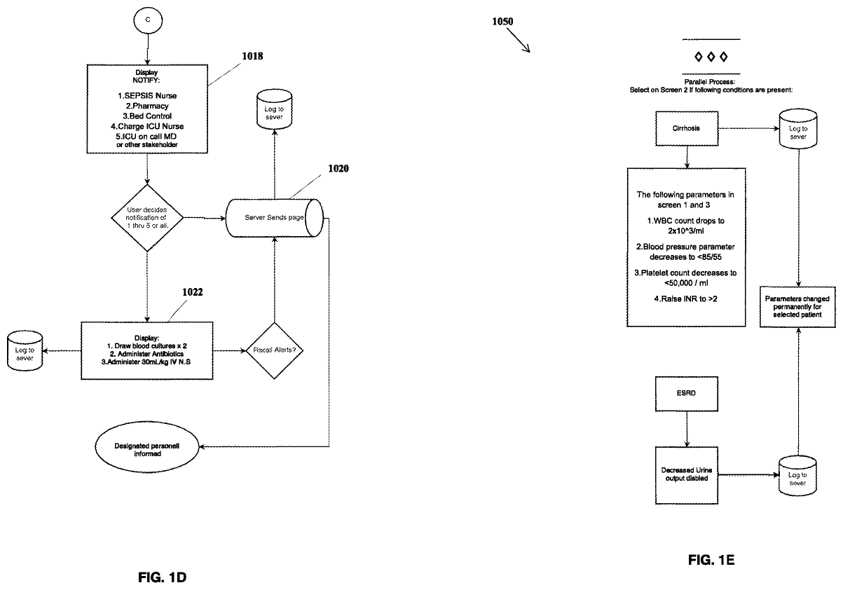

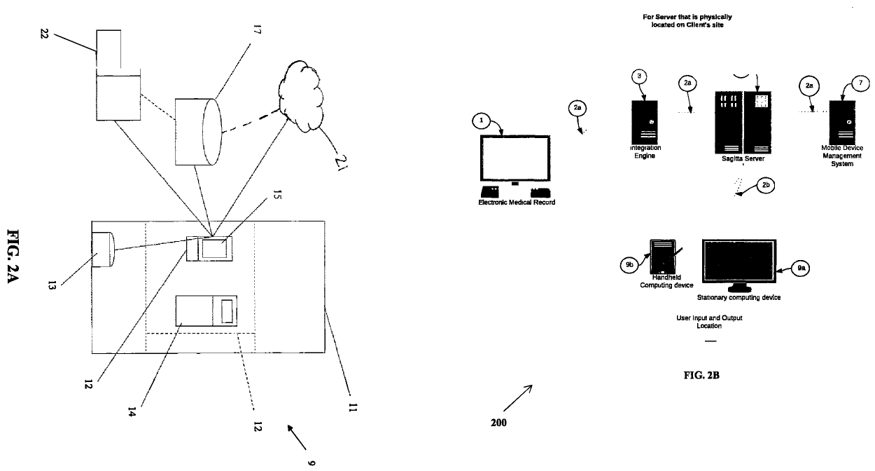

U.S. Pat. No. 10,592,637 (the '637 Patent), issued on Mar. 17, 2020, which claims the benefit of U.S. Provisional Application No. 62/096,754 filed on Dec. 24, 2014 (now expired) and U.S. Provisional Application No. 62/116,701 filed on Feb. 16, 2015 (now expired) provides relevant background hereto and is incorporated herein by reference for all purposes and made a part of the present disclosure. The following descriptions of FIGS. 1-6 are excerpted from the '637 Patent.

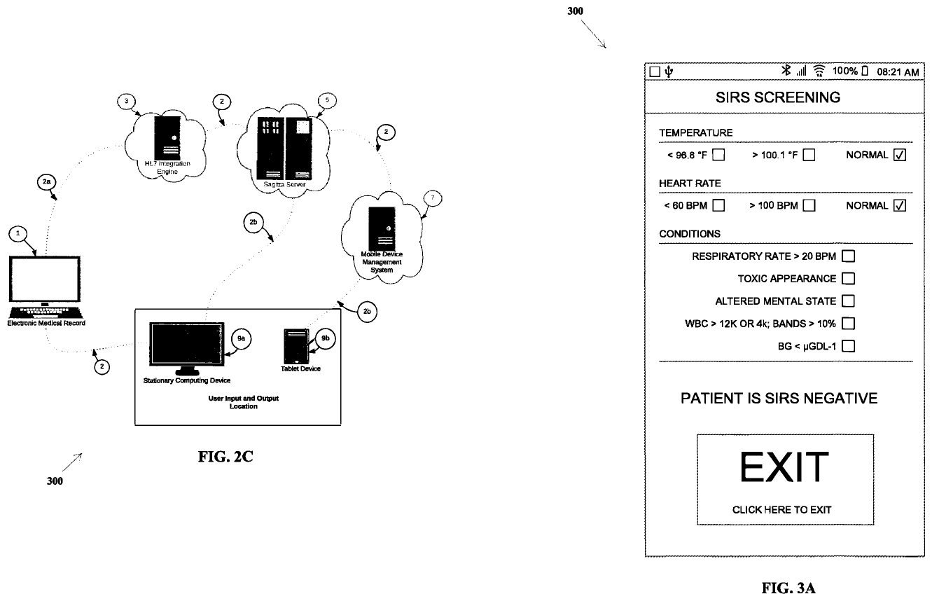

The simplified flowchart 100 of FIG. 1 presents, in basic terms, an exemplary method of screening for a medical condition, according to the present disclosure. The flowchart 1000 in FIGS. 1A-1E provides a more detailed description of an exemplary process or method of screening for a patient for sepsis that is particularly suited for implementation by a health care provider-user in a hospital or other health care environment wherein a patient is present (wherein like reference numerals are used to indicate like elements). The method according to either FIG. 1 or FIGS. 1A-1E is preferably and, more suitably, implemented utilizing a mobile or hand-held computing device storing executable programmable instructions and communicating with an electronic network (as discussed below in respect to FIGS. 2A-2C). The programmable instructions are executable to to generate and operate graphical user interfaces and to run screening algorithms for determining and/or outputting patient status in respect to the presence or probability of end organ damage due to infection or sepsis (or other medical condition). Such programmable instructions may be initiated by a health care provider-user entrusted with the computing device and preferably, at bedside.

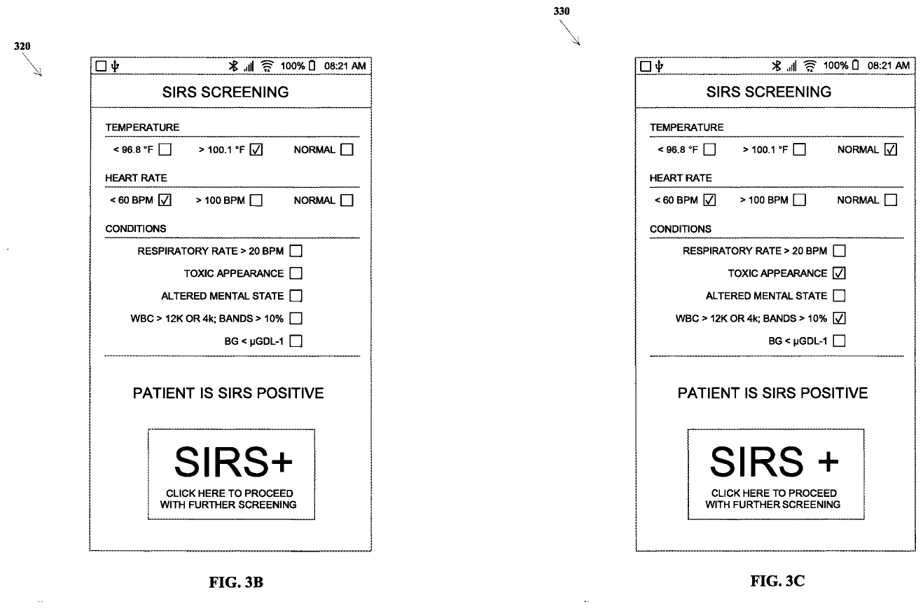

Referencing FIG. 1, the method is particularly suited for screening a patient for the potential for end-organ damage due to infection. In one aspect, the method is divided into three screening process stages 120,130,140 each of which may output one of two results (i.e., negative or positive), thereby advancing or diverting and ceasing the screening process. In another aspect, each of the three screening stages 120, 130, 140 is facilitated by availability of a hand-held computing device (storing executable programmable instructions) and a graphical user interface displayed thereon. Exemplary graphical user interfaces according to the disclosure are illustrated in FIGS. 3-5. Accordingly, the screening process stages 120, 130, 140 may be performed by a health care provider-user, at bedside, through operation of the computing device and the inputting of patient-related information via the graphical user interface.

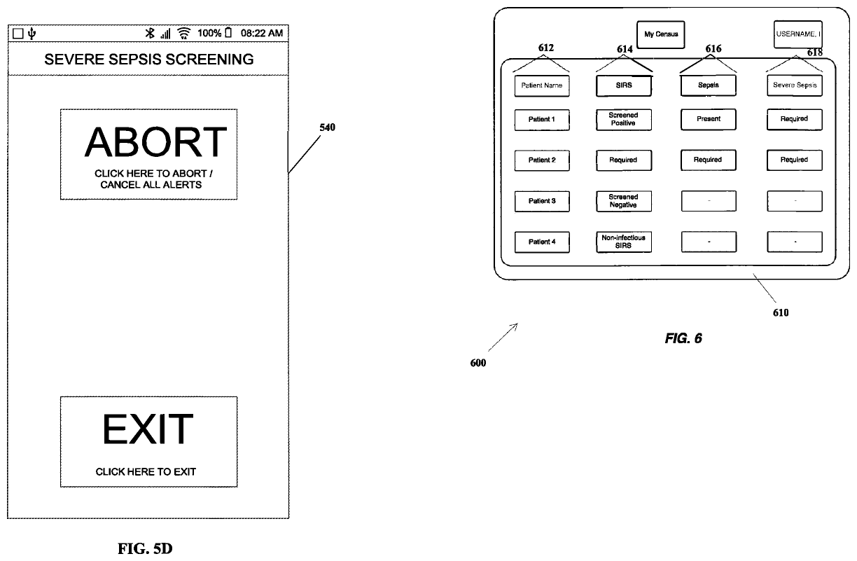

The process is commenced by identifying the patient 110, which preferably includes identifying and\or loading known patient information. Further, the patient may be registered on a system or network (see e.g., FIGS. 2A-2C), and identified on an electronic dashboard (see e.g. FIG. 6 and accompanying description below). In the first stage 120, patient information selected or collected provides input into an algorithm for determining the presence of Systematic Inflammatory Response Syndrome. The algorithm is run by initiating execution of the appropriate instructions via the graphical user interface. If a negative determination is made, the process outputs a patient status indicating the absence of Systematic Inflammatory Response Syndrome (121). As discussed below in more detail (see FIG. 2) in respect to Systematic Inflammatory Response Syndrome, a positive patient status is determined upon the detection and\or selection of two of a set of criteria. Among other things, the algorithm compares the user selections received on the graphical user interface with a screening criterion stored in memory. Otherwise, the process outputs a negative patient status (121). Optionally, the resultant patient status may be registered in the associated system or network (see e.g., FIGS. 2A-2C), associated with the patient in real-time, and indicated on the acuity dashboard (125).

With a positive Systematic Inflammatory Response Syndrome determination, the process advances to the second stage 130. In this stage 130, the health care provider, as user, provides clinical judgement-based input to the process and to a system algorithm for determining a “high” probability of end organ damage due to infection (noting that “high” in this context is not a relative value or degree but simply a title given for the positive determination). As discussed previously, the detection or selection of any one of a set of criteria determines a positive patient status for high probability of end organ damage due to infection. Otherwise, the absence of such detection or selection determines and outputs a False Positive patient status (131). Optionally, that resultant patient status may be registered in the associated system or network, associated with the patient in real-time, and indicated on the acuity dashboard (125).

With a positive determination of a high probability of end organ damage due to infection, the process advances to the third stage (130), in which the presence of severe or actual end organ damage due to infection is tested. In this stage, clinical parameters are provided, through user selections, as input to the appropriate algorithm. If any one of a set of criteria is detected or selected, end organ damage due to infection is determined. Otherwise, a negative patient status is outputted and the presence of Systematic Inflammatory Response Syndrome is identified with the patient (141). As before, that patient status may be registered in the associated system or network, associated with the patient in real-time, and indicated on the dashboard (125). In the alternative, upon the process output of the presence of end organ damage due to infection (142), that patient status may be registered in the associated system or network, associated with the patient in real-time, and indicated on the acuity dashboard (125). As a further option, the process may initiate one or more notification actions (150).

In one aspect, a system and method disclosed herein entails or incorporates the integration of a plurality of sources of patient-specific data and parameters related to the patient's medical conditions and status to create a knowledge base of patient data. These data sources may include: reports by health care providers on observations made at bedside; electronic medical records; and data from monitors and sensors (e.g., at the patient's bedside or at a remote location, or if directly attached to the patient). In another aspect or feature, the system or method provides for intelligently presenting patient data to the health care provider throughout an iterative analysis and, in which, the most current and accurate patient data is presented.

In yet another aspect or feature, a system and a method are described having the capability of identifying patients with Systemic Inflammatory Response Syndrome (i.e., a precursor to sepsis), providing the health care provider the information to determine if a patient has a significant suspicion or risk of infection or alternative explanations for non-infectious causes of Systematic Inflammatory Response Syndrome, and\or using the precursor information to guide the health care provider in an intelligent screening of the patient.

In one aspect, a computer implemented method of screening a patient with possible medical condition(s) is provided wherein the Application's algorithm(s) determine whether sufficient conditions are present to have a high likelihood of the targeted medical condition and then identifying that patient as having a high risk of the medical condition. The method further provides recommending an intervention plan for the patient including recruiting and assigning health care providers as needed for execution of the intervention plan, and iteratively analyzing patient data and recalibrating the intervention plans as necessary.

In another aspect or feature, a system or method is described providing hardware/software platform-independent delivery and a user interface customizable to the specifics of the situation including those of the user facility and that of the patient and the patient's medical condition. Further, the system or method features, in certain embodiments, diagnosis or patient screening algorithms that may be customized by integrating a human health care worker's job-specific work-flow into the system or method for real-time screening, diagnosis, and management of a given medical condition. For example, screening algorithms may be customized for an advanced practice registered nurse whose work flow differs from that of a bedside registered nurse in a typical hospital environment. The system or method may further include the capability of:
allowing supervising health-care providers to audit a patient population for whether or not screening has been performed;
allowing users the option to de-selects parts of the process algorithm to better reflect the unique requirements of a patient, thereby decreasing false positives and increasing positive predictive values;
deriving a real-time characterization of the patient census based on the patient's acuity of illness to prioritize and triage the patients identified with Systematic Inflammatory Response Syndrome or suspicion of infection or other medical conditions and to enable medical intervention based on comparative acuity between that patient and all others present in that facility in order to facilitate effective use of resources and staff;
effectively “learning” and adapting the system or method to the individual patient based on specifics of that patient including that patient's pre-existing medical conditions, current status, and evolving disease status; and
customizing sepsis screening or management of a given medical condition based on that patent's individual disease condition.

The system and Application according to the disclosure also provides a customizable, domain-specific user interface(s). The user interface is designed for health care providers and to lead them in an intuitive manner through a system or method of screening, diagnosing, and managing severe infections. The graphical user interface is further designed for efficient and effective data entry and analysis throughout screening, diagnosing, and managing of a patient. This point alone should reduce, if not eliminate, the typical initial tendency of a user toward a manual system or a workaround over an automated system (i.e., because of a perceived cumbersome and inflexible nature of the automated user interface).

The systems and processes disclosed herein also has the further capability of being integrated with industry standard electronic medical record systems, allowing health-care providers to assign patients with a given disease state to themselves and follow the patients' progress during the course of the shift. The system and process also allow for automatically updating the patient's electronic medical record to place orders for tests including but not limited to laboratory or radiographic tests.

Each of FIGS. 2A-2C depicts an exemplary system in which and by which the process(es) of FIG. 1 may be carried out (with like reference numerals used to indicate like elements). In one respect, the system comprises an input\output device, preferably a computing device, a server, a patient information database, and\or a cloud network. In a preferred embodiment, the computing device is a handheld computing device with user input means and output means. For example, the computing device may be a smart phone, tablet, notebook, laptop, or similar device. The input means includes a user responsive hardware interface having a keyboard or touch screen, as well as the standard array of communication ports and wireless interfaces. Preferably, the process description associated with FIG. 1 contemplates communicating through a voice activated and\or touch screen user interface. Suitable output means includes a conventional display and\or communications interface for connecting to a local or cloud network (including dedicated servers).

Implementation of the method in preferred embodiments provides one of several results or diagnoses. In one implementation, the method provides for or determines the apparent absence of Systemic Inflammatory Response Syndrome. In another or further implementation, the method provides a positive diagnosis of, and more specifically, a high probability of end organ damage due to severe infection. In yet another or further implementation, the method provides a positive diagnosis for end organ damage due to infection. In more specific implementations described below and illustrated by the flowcharts of FIGS. 1A-1E, the method provides a positive sepsis diagnosis, and more specifically, a high probability of sepsis or severe sepsis. Alternatively, the method may provide a false positive sepsis diagnosis. The method may, in fact, calculate a false positive sepsis diagnosis, just as an experienced and expert human health care provider may provide. It is expected, however, that implementation of the systems and methods according to the present disclosure will present a significantly smaller number of false positives than conventional systems and methods.

The flowchart 1000 of FIG. 1A-1E describes, in more detail, an exemplary method(s) of screening according to the present disclosure and specifically, screening a patient for sepsis according to a preferred embodiment. The method may be characterized as being implemented systemically or, as well, by medical personnel utilizing a hand-held or locally stationed computing device. The computing device includes and communicates with a resident (on-site at a facility hosting the patient) or remotely located processor(s) and memory storing programmable instructions executable to initiate or perform various steps in the process. These programmable instructions are packaged in a software application (Application) that can be initiated from or in the computing device. In any event, the computing device enables interactive graphical user interfaces to the software application responsive to input by the user and capable of projecting output displays and other activity generated by the Application, as described herein.

The process includes a series of user-prompted determinations which, if result in a series of affirmations, outputs a sepsis diagnosis. Each of the determinations is made through user interaction with three menu graphical user interfaces or screens—Screen 1, Screen 2, and Screen 3. Each Screen receives inputs (directly from user or from system) and runs an algorithm to output the determination. More specifically, each screen presents or prompts a set of user selections and the user responses or selections is received as patient data input to the algorithm. The method is initiated with activation of the computing device having a touch-based interface (1002). Typically, the user or users will have the device at hand and will be physically present in a hospital or clinic environment attending to a patient (although such physical proximity or direct contact is not necessarily required). Furthermore, the device may be connected to a cloud network either wirelessly or hard wired. Preferably, the user securely logins with a password or biometric scanning to maximize security.

In a preliminary step (1004), patient information is accessed and loaded for the patient. The patient's screening record may be accessed by: census retrieval from the server; scanning a patient wrist band; manual input; and accessing previous patient log. In another aspect of the present disclosure, when the patient is assigned to a specific health care provider, that health care provider's total group of patients is preferably displayed on an acuity-based electronic “dashboard.” An acuity dashboard, such as one depicted in FIG. 6 and discussed below, provides and identifies for the health care provider, among other things, the screening status of patients (e.g., whether patients have been screened or still require screening).

In one process application, the user proceeds to take the patient's vital signs and enters the information obtained via the device interface as input into the software application. Typically, the vital signs collected and inputted include:
1. Blood pressure—Systolic and Diastolic
2. Temperature
3. Respiratory Rate
4. Peripheral capillary oxygen tension/saturation
5. Blood Glucose level
   The application then pushes the data to the electronic medical record securely.

In a further aspect, the application also indicates to the user whether the value is normal or abnormal, for example, by presenting the screen background in red (as opposed to normal background color) or by employing some other alert. The Application typically provides an input screen by which the values collected may be entered by either turning a dial or moving a slider. Alternatively, the values may be selected on the touch interface and then manually entered. In any event, the Application significantly improves the process of data entry by eliminating the need to transcribe vital signs from the patient's bedside onto paper and then entering it into the computer.

In one aspect, the Application provides Screen 1 for a user-prompted determination based on physical measurements or measurements of the patient (1006). The procedure may determine, in the converse, the absence of Systematic Inflammatory Response Syndrome, thereby prompting the user to return to the main or native screen. As shown in the flowchart, Screen 1 presents to the user as queries and\or for selection by the user (or system), multiple possible patient condition parameters (Systematic Inflammatory Response Syndrome criteria). The user responses are received as input and the method runs an algorithm based on the input to determine patient status (1006). In accordance with the present disclosure, a user finding and menu selection of at least two of the presented variables leads to an intermediate diagnosis. The menu selections available on Screen 1 are printed in TABLE 1 below.

TABLE 1
SCREEN 1 SELECTIONS

| | |
|---|---|
| A. | Temperature greater than 100.1 less than 96.8; |
| B. | Heart rate greater than 100 bpm or less than 60 bmp; |
| C. | Respiratory rate > 20 breaths per minute; |
| D. | Having a new alteration of mental status or a toxic appearance; |
| E. | Laboratory values of white blood cell count greater than twelve |
| | thousand per ml or less than 4 thousand per ml or > 10% bands; |
| F. | Blood glucose less than 60 mg/dl or more than 160 mg/d; and |
| G. | None of the Above. |

If the user (human health care provider) does not observe any of the patient condition parameters as present in the patient, the user may select “none of the above” (as the Application input). The patient is effectively screened and the Application updates the system's knowledge base accordingly. The Application then displays “screening complete” complete. Upon the user hitting “OK”, the Application returns to the native screen. Further, if the Application's algorithm does not calculate the targeted condition being present (or the user provider selects the “none of the above” as above), the Application displays a “SYSTEMATIC INFLAMMATORY to RESPONSE SYNDROME ABSENT” screen (or equal) (1006a) before returning the user to the native screen. If the Application calculates, using the appropriate algorithm, that sufficient medical conditions are selected, the Systematic Inflammatory Response Syndrome screening criteria are met and the Application displays an alert stating “Systematic Inflammatory Response Syndrome present” (1006b). In the application according to FIGS. 1A-1E, the observation or selection of two or more condition parameters by the user indicates that the Systematic Inflammatory Response Syndrome screening criteria are met, thereby prompting display of the “Systematic Inflammatory Response Syndrome present” alert (1006b). Upon a user-activated prompt, the Application sends the server a label with a time stamp of “Systematic Inflammatory Response Syndrome present” (for that patient), which is then saved in the analytic server's database.

The Application now advances to Screen 2 (1010). Screen 2 enables the system to further adjust the sensitivity of screening based on numerous pre-test probability conditions. Each of the conditions in TABLE 2 below is provided as user selections on Screen 2.

TABLE 2
SCREEN 2 SELECTIONS-CLINICAL JUDGEMENT

| | |
|---|---|
| A. | Suspicion of Infection (Clinical judgment); |
| B. | Recent Antibiotic administration (Past Clinical Judgment); |
| C. | Recent procedure (increases probability of invasive infection); |
| D. | Presence of indwelling lines or catheters |
| | (increases probability of invasive infection; |
| E. | Positive blood cultures |
| | (increases probability of invasive infection); and |
| F. | None of the Above. |

As shown in FIG. 1B, a “none of the above” user selection results in the system presenting a False Positive diagnosis. A new user selection False Positive Screen is then presented with the menu of selections in TABLE 3 below (1010a).

TABLE 3
FALSE POSITIVE SCREEN SELECTIONS

| | |
|---|---|
| A. | OTHER; |
| B. | Benzodiazepine withdrawal; |
| C. | Neurolept malignant syndrome (NMS); |
| D. | drug fever; |
| E. | tumor fever; |
| F. | heat stroke; |
| G. | rhabdomyolysis; |
| H. | cardiac arrhythmias; and |
| I. | pulmonary embolism. |

The above menu provides possible reasons or sources for the False Positive diagnosis. If the user selects OTHER, the system provides a pop-up screen in which the user can manually insert a reason for the False Positive (1010c).

In one example, a “Subject A” is screened and results in a False Positive diagnosis. A reason for the False Positive, e.g., “Reason X”, is selected as discussed above and stored in the system (in the analytic server's database). If screening of Subject A is undertaken at a later time and arrives at Screen 1, and the same parameters as before are selected, the Application will present the query “Does the patient still have “Reason X”?.” This query substitutes the usual display, “Systematic Inflammatory Response Syndrome Present”, and may avoid the user having to go through Screen 2 and rendering another “FALSE POSITIVE” diagnosis. If “YES” is selected, then the Application displays the notification: “Patient previously tested False Positive for Systematic Inflammatory Response Syndrome due to “X” and prompts the user to further select either “CONTINUE” or “FINISH SCREENING.” If “Continue” is selected, the process advances to Screen 2 as before. If “FINISH SCREENING” is selected, the results are submitted to the server and the Application returns to native state.

In the alternative, if different parameters are selected on Screen 1 for Subject A, the program will display “Systematic Inflammatory Response Syndrome present.” On Screen 2 for “Subject A”, a clickable link is presented indicating that the patient had a previous False Positive Systematic Inflammatory Response Syndrome and when, activated, the “Reason X” will appear in a popup window. An “OK” button will also appear which, when selected, returns the process to Screen 2 for “Subject A.”

Returning to Screen 2, if one or more conditions A through E are selected, the Application will display the further diagnosis “High Probability Sepsis Present” in a window, which the user must activate (1010c). The user is then instructed to “Send Venous Lactate” with options “OK” and “Defer.” Upon user selection of “Send Venous Lactate,” the Nurse or other healthcare professional is prompted to send a blood sample of Venous lactate from the patient. Upon selection of either “OK” or “DEFER”, the server is sent a label with a time stamped “High Probability Sepsis Present” notice (for that patient). This information is also saved in the log.

As shown in FIG. 1C of the process, the user advances to Screen 3 after the “High Probability Sepsis Present” diagnosis (1014). At Screen 3, the Application runs an algorithm that receives user-selected clinical parameters as input and then screens the patient for signs and symptoms of Severe Sepsis (1014). The user is presented the menu of conditions for selection in TABLE 4 below.

TABLE 4
SCREEN 3 USER SELECTIONS-CLINICAL PARAMETERS

| | |
|---|---|
| A. | SBP < 90 mmHg (< 85 mmHg for Cirrhosis) |
| B. | SBP < 40 mmHg from baseline |
| C. | MAP < 65 mmHg |
| D. | Oxygen Saturation < 92% |
| E. | Increasing Oxygen requirement |
| F. | Decreased urine output or Creatinine > 2.0 mg/dL |
| G. | New Altered Mental Status |
| H. | Platelets < 100,000 |
| I. | Serum Lactic Acid Abnormal |
| J. | DIC/Petechiae or INR > 2; aPTT > 60 seconds |
| K | None of the Above |

Key:
SBP—Systolic Blood pressure
MAP—Mean Arterial Pressure
DIC—Dissiminated intravascular coagulation

If the user selects Option K (“None of the Above”), the Application displays in a popup window, “Patient is Systematic Inflammatory Response Syndrome” and “Please Repeat Screen in 2 hours” (1014a). With the user's approval, the Application sends the screening results information to the server and returns to native state. If the user opts to select “CANCEL”, the Application returns to Screen 3.

Otherwise, if the user selects of one or more of Options “B” through “J”, a diagnosis of “HIGH PROBABILITY OF SEVERE SEPSIS” is made and a corresponding display notification is provided (1014b). The Application then advances as shown in FIG. 1D and queries the user. Specifically, the user is presented a popup menu of available system notification activities (1018), including the following menu options:
1. SEPSIS Nurse
2. Pharmacy
3. Bed Control
4 Charge ICU Nurse
5. ICU on call MD

Upon selection of one or more of options, the system sends, via the server, the appropriate alphanumeric page to the designated personnel (1020). The Application then presents a menu (1022) displaying the following instructions:
1. Blood culture×2
2. Administer broad spectrum antibiotics within 60 minutes
3. Bolus 30 ml/kg N·S IV unless contraindicated

When the above instructions are fulfilled, the appropriate prompts on the display may be checked, and when checked (i.e. activating “OK”), the patient information is sent to the server. Furthermore, if the user wishes to recall the alert (after fulfilling instructions), an “ISSUE RECALL” notice may be prompted to send a second page cancelling the previous message. The Application then returns to native state.

The flowchart section (1050) of FIG. 1E represents a parallel process stemming from Screen 2 and accounting for patient conditions of Endstage Liver Disease/cirrhosis (ESLD) and Endstage Renal Disease (ESRD). This parallel process is initiated upon a user selection of “A” in Screen 3. The following parameters in Screens 1 and 3 are changed if the user indicates that the patient has Endstage Liver disease/cirrhosis (ESLD):
1. WBC count drops to 2×10\hat{ }3/ml;
2. Blood pressure parameter decreases to <85/55;
3. Platelet count decreases to <50,000/ml;
4. Dissiminated intravascular coagulation (DIC) selection is disabled; and
5. International Normalized Ratio raised to 3.

The following parameters in Screen 3 are changed if the user indicates that the patient has endstage renal disease (ESRD):
1. Decreased urine output selection is disabled.
2. Creatinine is ignored in all parts of the algorithm

The following table summarizes the adjustments to the Application Screens:

TABLE 5
ESLD/ESRD SCREEN OPTIONS

Nuances for ESLD: (see FIG 1E)
1. Option “A” will be decreased to < 85 mmHg
2. Option “H” will be decreased to < 50,000
3. Option “J” will be disallowed
Nuances for ESRD: (see FIG 1E)
Option “F” will be disallowed

In one embodiment, a system 9 according to the disclosure includes a computing device 10 as shown in FIG. 2 and as discussed above, configured for operation by one or more patient care personnel through a graphical user interface 15. Preferably, at certain stages of the process, the computing device 10 is operated within a patient care facility 11 (e.g., hospital), and more specifically, proximate a subject or patient 14 identified for screening (e.g., within a patient room 12). The patient care facility 11 may include a local network including a patient database 13 provided in communication with the computing device 10. The system 9 may further include a server 17 located remotely from the computing device 10, which may be configured with or as part of a cloud network 21. In this example, a database 22 is also included and similarly configured, as shown in FIG. 2. The database 22 may store patient-related or screening-related information inputted or logged as illustrated in the flowchart of FIG. 1.

Each of FIGS. 2B-2C depicts further preferred systems 200 in which and by which the process(es) of FIG. 1 may also be carried out, with like reference numerals used to indicate like elements. In FIG. 2B, the system 202 provides and\or incorporates a local network, while the system 202 of FIG. 2C provides and\or incorporates a cloud-based network. The system 200 includes one or more input\output devices, preferably handheld computing device 209a and\or stationary computing device 209b, a server 205, a patient information database 201, an integration engine 203, and a mobile device management system 207 that manages handheld computing devices 209b. In a preferred embodiment, the computing device is a handheld computing device with user input means and output means. For example, the computing device may be a smart phone, tablet, notebook, laptop, or similar device. The input means includes a user responsive hardware interface having a keyboard or touch screen, as well as a standard array of communication ports 202a and wireless interfaces 202b. It is contemplated that the processes described herein, including those described in respect to FIGS. 1 and 2, may communicate through a voice activated and\or touch screen user interface. Suitable output means includes a conventional display and\or communications interface for connecting to a local network or cloud network (including dedicated servers), as required.

Any number of commercially available mobile devices is suitable for use with the system and processes described. These include such devices having a touch-based interface, including such products referred to as iPad, iPhone, android tablets, and touch-based laptops. The mobile device is preferably connected to a wireless network for securely login. In an initial step, the patient's medical record is accessed by and through the device, and\or by scanning armband from camera, manual input, census retrieval for server, previous patient history from log on device. If the option is unavailable, the device and the process of information collection according to the present disclosure, presents the user the option to retry or else grey out option and employ any other three options to access the patient's data.

FIGS. 3A-3D provide illustrative examples of graphical user interfaces through which or by which certain steps of the process may be performed. These user interfaces include input and output means for communicating object data and information as discussed above in respect to the flowchart of FIG. 1. These user interfaces are particularly applicable for use on a handheld device such as a smartphone or digital tablet. The user interface 300 of FIG. 3A may correspond to the “Load Patient Information” screen or step in FIG. 1, which is used to initiate screening. The user interfaces 320, 330 of FIG. 3B or 3C may correspond with or provide Screen 1 of the process. Notably, screen 330 on FIG. 3C indicates certain patient parameters inputted for the Anonymous Patient. The system then outputs a notice to the user based on the indications in FIG. 3C. As discussed above, the presence of two Systemic Inflammatory Response Syndrome criteria/parameters triggers a “Systemic Inflammatory Response Syndrome Patient” notice and display.

Table 6 provides additional, exemplary graphical user interfaces suitable for use with the systems and methods described herein, including the method/process according to FIGS. 1A-1E.

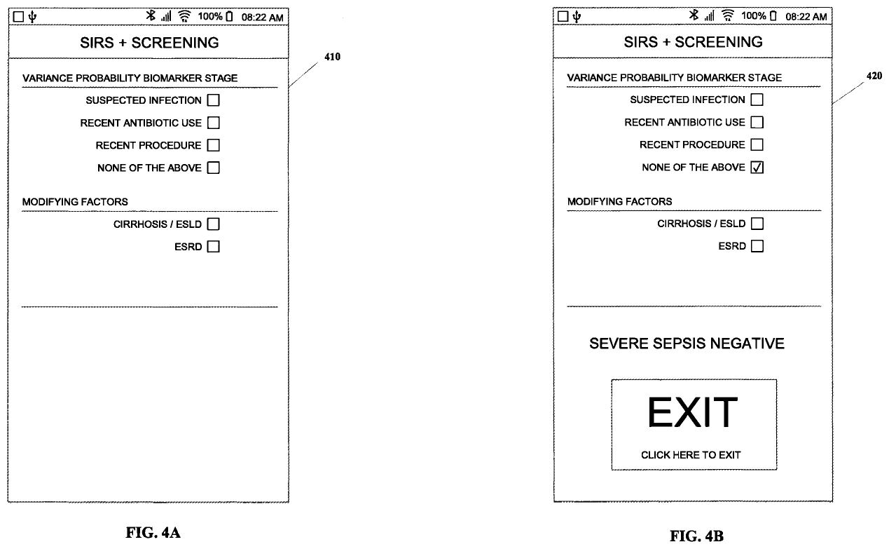

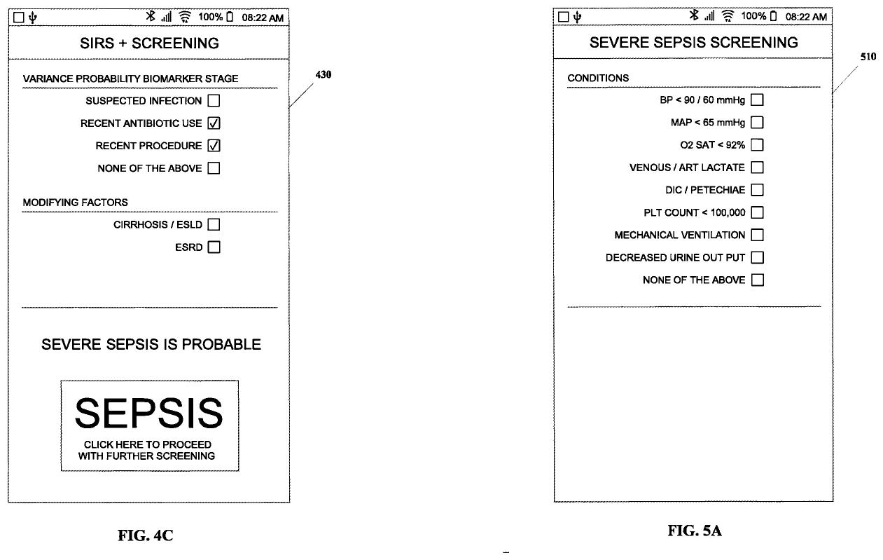

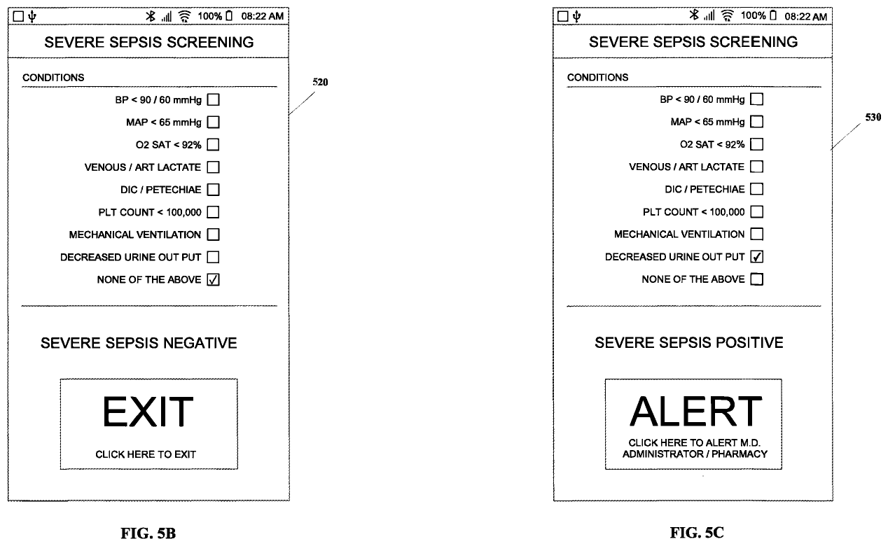

TABLE 6
GRAPHICAL USER INTERFACES

| | |
|---|---|
| 1. | “SCREEN 2” 410 (FIG. 4A) (with user selection options presented) |
| 2. | Output Screen 420 (FIG. 4B) (with one user selection inputted) |
| 3. | Output Screen 430 (FIG. 4C) (with two affirmative user selections |
| | prompting positive patient status - high probability of sepsis) |
| 4. | “SCREEN 3” 510 (FIG. 5A) |
| 5. | Output Screen 520 (FIG. 5B) |
| 6. | Output Screen 530 (FIG. 5C) |
| 7. | Abort/Exit Screen (FIG. 5D) |

The presently described systems and processes aims to identify patients with a high risk of end-organ damage due to severe medical conditions and further, provide a health care provider with decision support. This may entail providing pre-populated vital signs or calculating shock index to determine if the patient has SIRS, where shock index is calculated by a standard industry practice of dividing the heart rate by the systolic blood pressure, and also pre-populating laboratory and other pertinent clinical data. Preferably, this support eliminates the need for the health care provider to comb through data that is not pertinent to the patient's severe medical condition. The method of decision support may also include explaining and documenting common medical factors that, previously, would have led to misdiagnosing the severe medical condition, so as to reduce false positives and increase positive predictive value of the computing system iteratively. Support recommendations are maintained in the patient's electronic medical record and in the database.

For example, a patient may suffer end-organ damage (severe medical condition) due to infection, but the patient originally presents with a non-infectious cause for SIRS (false positive for infection) and, correctly, is not given antibiotics. Later, the patient develops SIRS that is from an infectious cause and at this later date has a delay in administration of antibiotics due to the health care provider not having noticed the appropriate markers of a new and developing condition (that was not present initially). To illustrate further, a patient may have a fever to 101 F and has a tachycardia with a heart rate of 120, and is diagnosed as having SIRS. The patient is considered as being free of any clinical signs or focus of infection on laboratory or radiographic studies, according to the experienced health care provider. The patient has an inflammation of the pancreas (pancreatitis) that frequently causes fever and tachycardia. The nurse marks that the patient has non-infectious SIRS secondary to pancreatitis. A few days later, the patient develops an elevated white blood cell count with elevated “band forms” (immature white blood cells—an indicator of new or developing infection) and has a decrease in blood pressure (resulting in a high shock index). The patient is correctly identified as having a second SIRS state which is different from the initial state. The nurse is able to effectively recruit assistance from a physician to order antibiotics and a pharmacist to bring those antibiotics to bedside in a time manner.

With current technology, this patient would have continued for more than eight hours without appropriate treatment due to an anchoring heuristic resulting in an increased mortality and harm to the patient (literature shows that mortality increases by 7% for every hour of delayed administration of antibiotics ref: Kumar et al, 2007.) This present system and process minimizes false positives and maintains that information so that if the patient develops infection-related SIRS and presents legitimate symptoms at a later time, the patient is correctly diagnosed with infection-related SIRS.

Implementation of the presently disclosed systems and processes with conventional screening and treatment processes currently employed is expected to achieve certain benefits, including reduction in mortality rates, cost, and lengths of patient stay in the hospital. There is also an expected reduction in response time to a patient who meets criteria based on the system's alert protocols. This prevents escalation from sepsis to severe sepsis and/or septic shock for “at-risk” patients after hospital admission due to delay in treatment.

There is also an expected improvement in data management by using templated notes-ability to import sepsis log and post to patient's medical record to complete the heath care provider's documentation. Furthermore, quality improvement mechanisms are made more agile by real time data reporting, which allows for on-the-fly review of data (no need for onerous chart review) and shorter plan-do-study-act (PDSA)cycles for quality improvement. This is especially important since adherence to sepsis bundles is reportable starting September 2015

There may also be numerous benefits to the health care provider. For example, the presently disclosed systems and processes should aid in preventing “Algorithm Agnosia”—a condition in which the health care provider discounts or fails to recognize a true positive event due to repeated prior false alarms. Implementation of the presently disclosed system and process should also aid in process normalization within the hospital by reducing variations of care, decrease telemetry utilization by frequently screening high risk patients on non-telemetry floors, and prevent inaccurate assessments of patients on transferring from one level of care to another (e.g., emergency room to the floor instead of emergency room to intensive care unit).

The following is a description of a system by which data is extracted from the electronic medical record and then independently processed to achieve desired output.

### Examples

1. Sepsis Screening: Output data of whether patient is septic or not based on Systemic Inflammatory Response Syndrome plus lab/radiology input from EMR

2. Clinical optimization of end-stage heart failure patients for assist device implantation or heart transplantation—data processing uses analysis to determine what next steps the clinician must take to optimize the patient for assist device implant (e.g., if an obese patients who was previously excluded from transplant for morbid obesity, starves themselves and BMI drops from 30-28 between clinic visits but pre-albumin level also drop the program will indicate to the provider that a nutrition evaluation is necessary and that the clinically significant drop in BMI indicates a deterioration in health rather than the person getting closer to being a candidate for transplant.

3. Transfer evaluation for higher level of care: in a non-tertiary care hospital the entire hospital census is analyzed on a real-time basis and an acuity score is calculated for each patient.

Based on the acuity score, each patient will be evaluated for benefit of transfer to a higher level of care, e.g., a patient with elevated bilirubin, elevated AST/ALT, INR >2.0 and platelet count <100,000 will benefit from transfer to a higher level of care for liver transplant evaluation. This data will be presented to the attending physician with the option to refer the patient to a medical center where a hepatologist is available. If the doctor accepts the face-sheet and insurance details are automatically pushed to both transfer centers (accepting and sending facilities). The on-call consultant and hospitalists are notified electronically and clinical data is presented to them. If the patient is deemed appropriate for transfer a doctor to doctor conversation is initiated automatically via the transfer center between all three providers. This system streamlines the existing workflow and ensures that patients who would benefit from higher level care are provided the best care available.

The above provide some examples. It should be apparent however to one skilled in the art that a framework is provided that allows clinicians to build any sort of query algorithm to process data on a real-time basis.

Sepsis is a general term used to describe a syndrome that occurs when the body's immune system overreacts to a local infection, resulting in uncontrolled system-wide inflammation. Undiagnosed sepsis is a significant threat to patients' well-being, as well as a major drain on the time, energy and resources of healthcare institutions. Several independent studies have shown that screening for sepsis reduces mortality rates in hospitalized patients. Use of the presently described systems, process, and graphical user interfaces will aid in streamlining sepsis care in acute care facilities and decreasing mortality rates, costs and length of stay. A protocol utilizing the presently disclosed systems and processes implements a sustainable sepsis screening based on related algorithms for acute care hospitals. The protocol uses real-time data import from several sources and interacts directly with team members and processes including: the bedside nurse, nursing assistant, pharmacy, the hospital sepsis team, bed control. As statistically based measurement and standardization are often cited as the first steps to improving quality, the present systems and processes lend themselves to such improvements.

In preferred embodiments, the system and process utilized tablet-based bedside software. As it is server-linked, the tablet device provides real-time clinical decision-making support. By providing real-time data to hospitals on acuity, incidence of Systemic Inflammatory Response Syndrome/sepsis and severe sepsis, one can control positive predictive value. The systems and processes allow, therefore, for more agile processing and customization to individual facilities. Sepsis screening is tailored for patients with pre-existing organ system pathology. With the presently disclosed system and processes, including the Application, one can modify individual variables to account for end-stage liver disease (ESLD); atrial fibrillation; heart failure; end-stage renal disease (ESRD), ventricular assist devices (LVAD and RVAD), balloon pumps and ventilators.

A suitable mobile or hand-held device for use with the presently disclosed systems and processes is any one of a number of commercially available mobile devices having a touch-based interface, including such products referred to as iPad, iPhone, android tablets, and touch-based laptops. The device is preferably connected to wireless network to securely login. In an initial step, the patient's medical record is accessed by and through the device, and further by scanning armband from camera, manual input, census retrieval for server, previous patient history from log on device. In a typical operation, the camera input is a preferred first choice for access. If the option is unavailable, the device and the process of information collection according to the present disclosure, presents the user the option to retry or else grey out option and employ any other three options to access the patient's data.

FIG. 6 depicts an exemplary graphical user interface 600 embodying and illustrating one example of an acuity dashboard 610 according to the present disclosure. The graphical user interface 600 may be provided on a handheld computing device, other mobile device, laptop, desktop, terminal, or other computing device preferably connected and in communication with the system(s) described herein, including those in FIGS. 2A-2C. Typically, the acuity dashboard 610 is unique to a health care provider-user and provides for that health care provider his\her patient census (groups of associated patients). In the acuity dashboard 600 of FIG. 6, information is conveniently provided in several columns, including a column 612 for Patient Name (i.e., patients assigned to the health care provider-user). The remaining columns (614, 616, 618) provide the patient's screening status for each of Systematic Inflammatory Response Syndrome, Sepsis, and Severe Sepsis. The patient status information is, of course, preferably delivered and updated, in real-time, by and from operation of the hand-held devices and Screens 1-3 described in respect to FIG. 2, for example.

Referring to the acuity dashboard of FIG. 6, Patient 1 is shown having been screened (using Screens 1 and 2) to determine the presence of Systematic Inflammatory Response Syndrome and the presence of Sepsis. Patient 1 is also shown as (still) requiring screening (using Screen 3) for Severe Sepsis, as prompted by a positive result in the screening sub-process using Screen 2. In contrast, patient status for Patient 3 shows that the patient as being screened negative for Systematic Inflammatory Response Syndrome and thus not requiring further screening. Similarly, Patient 4 is shown not requiring further screening as well due to a scenario, for example, described below is Usage Scenario 4.

The following are exemplary Scenarios wherein an acuity dashboard and one or more processes as previously described are embodied and utilized in an Application. The process steps may be performed by user(s) with a handheld device or other computing device as previously discussed, and by or through a system or network as also previously discussed. The hardware system or network and\or software application is referred to as the Application for purposes of description.

Usage Scenario 1: New Patient Entered into System and Application Calculates a High Probability of Sepsis.

Health Care Provider starts Application on device.

Health care Provider accesses hospital census from Application and adds new patient to their list (this patient is not yet on the list of previously screened patients).

The initial acuity dashboard screen presents the following comparative information for the Health Care Provider to consider, where the comparison is being made relative to other patients on that user's list of patients while calculating probability for that individual patient:
a. Presence of high probability Systematic Inflammatory Response Syndrome by displaying “Required Screening” under Systematic Inflammatory Response Syndrome column
b. Presence of high probability of infection by evaluating surrogate markers of infection by displaying “Required Screening” under Sepsis column
c. Indicating whether or not biomarker labs are required or have already been sent in the “Biomarker” column by indicating “required” or “sent” or “resulted” respectively
d. Presence of high probability of end-organ damage due to infection by displaying “Required Screening” under Severe Sepsis column

The Health Care Provider then selects the patient to further evaluate the reasons for Systematic Inflammatory Response Syndrome, presence of infection and presence of end organ damage due to infection by initiating the screening process.

Application calculates based on the user's input and validation of the information presented via the user interface to determine the presence of Systematic Inflammatory Response Syndrome, Infection and end organ damage due to infection.

The dashboard screen now presents to the Health Care Provider the following information:
e. Presence of verified high probability of Systematic Inflammatory Response Syndrome by displaying “Present” under Systematic Inflammatory Response Syndrome column
f. Presence of infection being the cause of Systematic Inflammatory Response Syndrome by displaying “Present” under Sepsis column
g. If the lab test has been sent it will display “sent” or “resulted” depending on if the lab test is available for review (resulted) or is still under processing (sent). It will further determine if the lab value is “normal” or “abnormal” and will display the same under column “Biomarker”.
h. Presence of end-organ damage caused by infection by displaying “Present” under the Severe Sepsis column

The Health Care Provider then sends alerts stating “Sepsis present” to the other health care providers involved in the patient's care via Application which may interface to existing pagers, smartphones or text-based messaging devices.

Usage Scenario 2: New Patient Entered into System and Application Calculates a Low Probability of Sepsis Because the Patient does not have Sepsis (True Negative).
1. Health Care Provider starts Application on device.
2. Health Care Provider accesses hospital census from Application and adds new patient to their list (this patient is not yet on the list of previously screened patients).
3. The initial acuity dashboard screen presents the following comparative information for the Health Care Provider to consider, where the comparison is being made relative to other patients on that user's list of patients while calculating probability for that individual patient:
   a. Presence of low probability Systematic Inflammatory Response Syndrome by displaying “Absent” under Systematic Inflammatory Response Syndrome column
   b. Absence of surrogate markers of infection by displaying “-” under Sepsis column
   c. Indicating that biomarker labs are not required by displaying “-” in the “Biomarker” column
   d. Presence of low probability of end-organ damage due to infection by displaying “-” under Severe Sepsis column
4. The Health Care Provider in this case will not further screen this patient and thereby not require allocation of time or other resources to evaluate the patient.

Usage Scenario 3: Existing Patient Updated in System and Application Calculates a High Probability of Sepsis.
1. Health Care Provider starts Application on device.
2. Health Care Provider reviews list of existing patients they have previously assigned themselves
3. The initial acuity dashboard screen presents the following comparative information for the Health Care Provider to consider, where the comparison is being made relative to other patients on that user's list of patients while calculating probability for that individual patient who previously did not have sepsis:
   a. Presence of high probability Systematic Inflammatory Response Syndrome by displaying “Required Screening” under Systematic Inflammatory Response Syndrome column
   b. Presence of high probability of infection by evaluating surrogate markers of infection by displaying “Required Screening” under Sepsis column
   c. Indicating whether or not biomarker labs are required or have already been sent in the “Biomarker” column by indicating “required” or “sent” or “resulted” respectively
   d. Presence of high probability of end-organ damage due to infection by displaying “Required Screening” under Severe Sepsis column
4. The Health Care Provider then selects the patient to further evaluate the reasons for Systematic Inflammatory Response Syndrome, presence of infection and presence of end organ damage due to infection by initiating the screening process.
5. Application calculates based on the user's input and validation of the information presented via the user interface to determine the presence of Systematic Inflammatory Response Syndrome, Infection and end organ damage due to infection.
6. The dashboard screen now presents to the Health Care Provider the following information:
   a. Presence of verified high probability of Systematic Inflammatory Response Syndrome by displaying “Present” under Systematic Inflammatory Response Syndrome column
   b. Presence of infection being the cause of Systematic Inflammatory Response Syndrome by displaying “Present” under Sepsis column
   c. If the lab test has been sent it will display “sent” or “resulted” depending on if the lab test is available for review (resulted) or is still under processing (sent). It will further determine if the lab value is “normal” or “abnormal” and will display the same under column “Biomarker”.
   d. Presence of end-organ damage caused by infection by displaying “Present” under the Severe Sepsis column
7. The Health Care Provider then sends alerts stating “Sepsis present” to the other health care providers involved in the patient's care via Application which may interface to existing pagers, smartphones or text-based messaging devices.

Usage Scenario 4: New Patient Updated in System and Application Calculates a Low Probability of Sepsis Because the Patient has Symptoms that could Cause a False Positive.
1. Health Care Provider starts Application on device.
2. Health Care Provider accesses hospital census from Application and adds new patient to their list (this patient is not yet on the list of previously screened patients).
3. The initial acuity dashboard screen presents the following comparative information for the Health Care Provider to consider, where the comparison is being made relative to other patients on that user's list of patients while calculating probability for that individual patient:
   a. Presence of high probability Systematic Inflammatory Response Syndrome by displaying “Required Screening” under Systematic Inflammatory Response Syndrome column
   b. Absence of high probability of infection by absence surrogate markers of infection by displaying “Required Screening” under Sepsis column
   c. Indicating whether or not biomarker labs are required or have already been sent in the “Biomarker” column by indicating “required” or “sent” or “resulted” respectively
   d. Absence of high probability of end-organ damage due to infection by displaying “-” under Severe Sepsis column
4. The Health Care Provider then selects the patient to further evaluate the reasons for Systematic Inflammatory Response Syndrome, presence of infection and presence of end organ damage due to infection by initiating the screening process.

5. Application calculates based on the user's input and validation of the information presented via the user interface to determine the presence of Systematic Inflammatory Response Syndrome, Infection and end organ damage due to infection. In this case the user determines that there is no infection present due to the absence of surrogate markers of infection. User selects a reason for Systematic Inflammatory Response Syndrome that is not related to infection.
6. The dashboard screen now presents to the Health Care Provider the following information:
   a. Presence of Non-infectious cause of Systematic Inflammatory Response Syndrome by displaying “Non-Infectious Systematic Inflammatory Response Syndrome” under Systematic Inflammatory Response Syndrome column
   b. Absence of infection being the cause of Systematic Inflammatory Response Syndrome by displaying “Absent” under Sepsis column
   c. If the lab test has been sent it will display “sent” or “resulted” depending on if the lab test is available for review (resulted) or is still under processing (sent). It will further determine if the lab value is “normal” or “abnormal” and will display the same under column “Biomarker”.
   d. Absence of end-organ damage caused by infection by displaying “-” under the Severe Sepsis column
7. The Health Care Provider by selecting the reason for “Non-Infectious Systematic Inflammatory Response Syndrome” via this process modifies the parameters of that discrete patient in order that their Systematic Inflammatory Response Syndrome alert will not trigger for the same reasons as they triggered the initial alert. By this manner future false positives are avoided.

Usage Scenario 5: Existing Patient Updated in System and Application Calculates a Low Probability of Sepsis Because the Patient has Symptoms that could Cause a False Positive.
1. Health Care Provider starts Application on device.
2. Health Care Provider reviews list of existing patients they have previously assigned themselves
3. The initial acuity dashboard screen presents the following comparative information for the Health Care Provider to consider, where the comparison is being made relative to other patients on that user's list of patients while calculating probability for that individual patient who previously did not have sepsis:
   a. Presence of high probability Systematic Inflammatory Response Syndrome by displaying “Required Screening” under Systematic Inflammatory Response Syndrome column
   b. Absence of high probability of infection by absence surrogate markers of infection by displaying “Required Screening” under Sepsis column
   c. Indicating whether or not biomarker labs are required or have already been sent in the “Biomarker” column by indicating “required” or “sent” or “resulted” respectively
   d. Absence of high probability of end-organ damage due to infection by displaying “-” under Severe Sepsis column
4. The Health Care Provider then selects the patient to further evaluate the reasons for Systematic Inflammatory Response Syndrome, presence of infection and presence of end organ damage due to infection by initiating the screening process.
5. Application calculates based on the user's input and validation of the information presented via the user interface to determine the presence of Systematic Inflammatory Response Syndrome, Infection and end organ damage due to infection. In this case the user determines that there is no infection present due to the absence of surrogate markers of infection. They select a reason for Systematic Inflammatory Response Syndrome that is not related to infection.
6. The dashboard screen now presents to the Health Care Provider the following information:
   a. Presence of Non-infectious cause of Systematic Inflammatory Response Syndrome by displaying “Non-Infectious Systematic Inflammatory Response Syndrome” under Systematic Inflammatory Response Syndrome column
   b. Absence of infection being the cause of Systematic Inflammatory Response Syndrome by displaying “Absent” under Sepsis column
   c. If the lab test has been sent it will display “sent” or “resulted” depending on if the lab test is available for review (resulted) or is still under processing (sent). It will further determine if the lab value is “normal” or “abnormal” and will display the same under column “Biomarker”.
   d. Absence of end-organ damage caused by infection by displaying “-” under the Severe Sepsis column
7. The Health Care Provider by selecting the reason for “Non-Infectious Systematic Inflammatory Response Syndrome” via this process modifies the parameters of that discrete patient in order that their Systematic Inflammatory Response Syndrome alert will not trigger for the same reasons that triggered the initial alert. By this manner future false positives are avoided.

Usage Scenario 6: Existing Patient Updated in System and Application Calculates a Low Probability of Sepsis Because the Patient does not have Sepsis (True Negative).
1. Health Care Provider starts Application on device.
2. Health care provider reviews list of existing patients they have previously assigned themselves
3. The initial acuity dashboard screen presents the following comparative information for the Health Care Provider to consider, where the comparison is being made relative to other patients on that user's list of patients while calculating probability for that individual patient:
   a. Presence of low probability Systematic Inflammatory Response Syndrome by displaying “Absent” under Systematic Inflammatory Response Syndrome column
   b. Absence of surrogate markers of infection by displaying “-” under Sepsis column
   c. Indicating that biomarker labs are not required by displaying “-” in the “Biomarker” column
   d. Presence of low probability of end-organ damage due to infection by displaying “-” under Severe Sepsis column
4. In this scenario, the Health Care Provider will not further screen the patient and will not require allocation of time or other resources for evaluating the patient.

### Infectious Disease Screening

Some embodiments disclosed herein are systems, methods, apparatus, and graphical user interfaces for screening, managing, diagnosing and\or treating a patient with an infectious disease. In some such embodiments, the infectious disease is a viral infection, such as a coronavirus (e.g., COVID-19) or influenza. The method can be a computer-implemented method and the system can include a handheld or locally stationed device connected with one or more processors.

In some embodiments, components of the systems, methods, apparatus, and graphical user interfaces discussed above with reference to FIGS. 1-6 for screening for sepsis and other conditions may be modified and implemented for use in screening for infectious diseases.

In some embodiments, patient data is requested and/or received and/or input into the graphical user interface for analysis by the system. The patient data is analyzed by the system for indications of a probability infection. Some exemplary patient data includes: patient age; whether the patient has had close, in-person contact with another person that has been diagnosed with the infectious disease; whether the patient has traveled internationally (e.g., within the past 14 days); whether the patient is feeling sick; what symptoms the patient is experiencing (e.g., fever), if any; whether the patient lives in a nursing home or similar facility; whether the patient is a first responder or health care worker; and the patients geographical location. Requests for such patient data can be presented in a GUI to the patient, with the GUI configured to provide the patient with the ability to input answers to the requests. In some embodiments, patient interacts with the GUI via the internet (e.g., a web-based tool). For example, the GUI may be presented on a website visited by the patient using a computer. In some embodiments, the patient has the option to not answer at least some of the questions. For example, the patent may choose not to provide a geographical location. Each patient data may be associated with an increased or decreased probability of having the infectious disease. For example, not having a symptom, such as a fever, can decrease the assessed probability of that patient having COVID-19. While, having a symptom, such as a fever, can increase the assessed probability of that patient having COVID-19. In some embodiments, the patient data is entered and/or maintained anonymously. In some embodiments, if a patient is determined to have COVID-19 or to have a probability of having COVID-19 that is above a threshold probability, the system, apparatus, and/or graphical user interface can direct the patient to contact a local health department or call 911. For example, the GUI can present a link to place such a call or can provide a number to call. In some embodiments, the requests for patient data can take less than two minutes for the patient to complete.

In some embodiments, the systems, methods, apparatus, and graphical user interfaces disclosed herein are usable by an organization to monitor members of that organization. For example, an employer may use the systems, methods, apparatus, and graphical user interfaces disclosed herein to monitor the status of employees with regards to the infectious disease. This can allow the employer to prevent or at least reduce the likelihood of an outbreak of the infectious disease occurring within that business organization. For example, when an employee of the business organization is identified as having COVID-19, or is identified as having a probability of having COVID-19 that is above a threshold probability, then the employer can implement quarantining of that employee from other employees. a fever. In some embodiments, such as when the patient data is collected anonymously, the patient data can inform the organization of the transmission and severity of the virus in certain locations without informing the organization of the individual that has the virus.

The systems, methods, apparatus, and graphical user interfaces disclosed herein can be configured for use by a specific organization (e.g., a specific business, non-profit, or government entity). The systems, methods, apparatus, and graphical user interfaces disclosed herein can be configured for general use by the general public. The systems, methods, apparatus, and graphical user interfaces disclosed herein can be configured for use in multiple, different languages.

In some embodiments, use of the systems, methods, apparatus, and graphical user interfaces disclosed herein can decrease emergency room (ER) overcrowding and reduce health care workers' potential exposure to the virus by identifying the virus before the patient directly interacts with health care workers. One exemplary use of the systems, methods, apparatus, and graphical user interfaces disclosed herein is in the screening of homeless people, providing for the determination of whether individual homeless people require testing, quarantining, or health care.

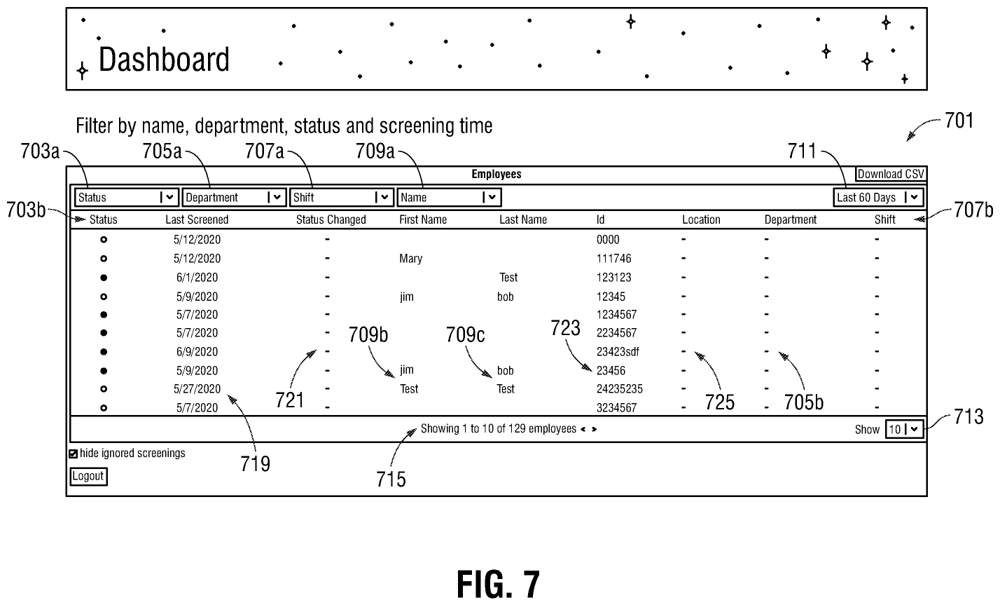

FIGS. 7-16 depicts exemplary portions of a GUI for use in screening for and tracking the occurrence of COVID-19. While shown and described in reference to COVID-19, the systems, methods, apparatus, and graphical user interfaces disclosed herein can be used for other infectious diseases. Also, while referred to as a “patient”, the individuals are not necessarily patients, and may be employees or members of an organization.

FIG. 7 depicts dashboard 701. Dashboard 701 allows for the tracking of the COVID-19 screening multiple patients. Dashboard 701 provides the ability to filter through patient data by patient name, department (e.g., for organizations with multiple departments), patient status (e.g., infected, not infected), and screening time.

Dashboard 701 includes status filter 703a, which provides a user of the dashboard 701 the ability to sort patients via the status of that patient. Dashboard 701 includes status column 703b, which lists and identifies a status of the patients. For example, the green light may indicate that a patient is considered to not have COVID-19, the red light may indicate that a patient is considered to have COVID-19, and the yellow light may indicate that a patient is considered to be at risk of having or contracting COVID-19.

Dashboard 701 includes department filter 705a, which provides a user of the dashboard 701 the ability to sort patients via the department of that patient. Dashboard 701 includes department column 705b, which lists and identifies a department of the patients.

Dashboard 701 includes shift filter 707a, which provides a user of the dashboard 701 the ability to sort patients via the shift of that patient (e.g., the shift that the patient works). Dashboard 701 includes shift column 707b, which lists and identifies a shift of the patients.

Dashboard 701 includes name filter 709a, which provides a user of the dashboard 701 the ability to sort patients via the name of that patient (e.g., the shift that the patient works). Dashboard 701 includes name columns 709b and 709c, which lists and identifies the first and last names of the patients, respectively.

Dashboard 701 includes a time frame filter 711, which provides a user of the dashboard 701 the ability to view patient data within a certain time frame (e.g., the last 60 days).

Dashboard 701 includes a number of results selector 713, which provides a user of the dashboard 701 the ability to view a desired number of patient's data (e.g. 10 per page). Dashboard 701 includes number of results identifier 715, which identifies the number of patients present in the GUI, as well as the total number of results (e.g., “showing 1 to 10 of 129 employees).

Dashboard 701 also includes a last screened column 717 indicating when the patient was last screened for COVID-19; a status changed column 719 indicating if the status of the patient has changed (e.g., changed from not infected to infected); an ID column 723 indicating a unique ID for the patient; and a location column 725 indicating a location of the patient.

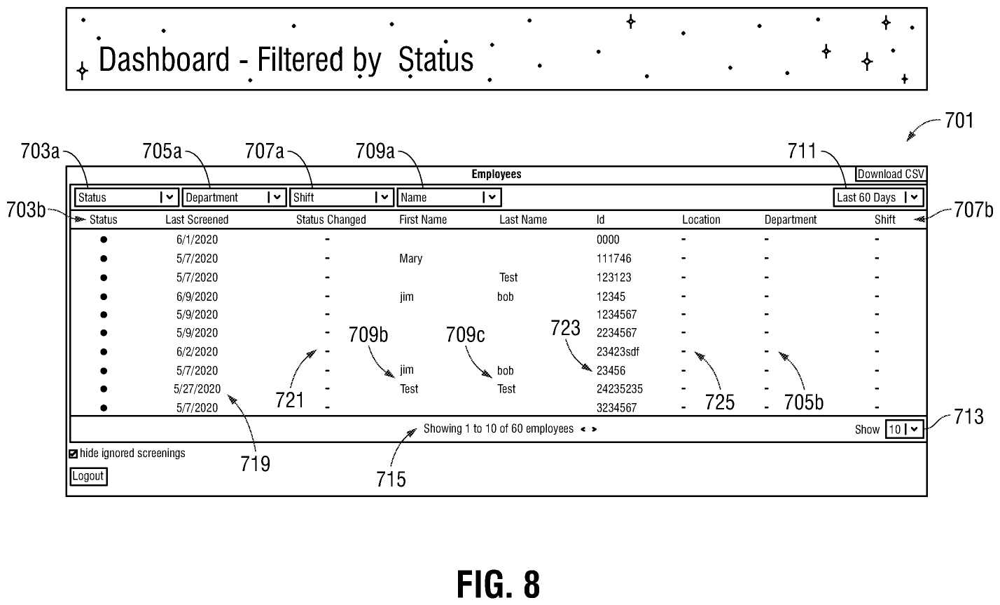

FIG. 8 depicts dashboard 701, with a user of the dashboard 701 having selected to filter the list of employees such that only those with a “red” status are presented. In addition, multiple filters may be selected concurrently to further narrow a desired list of results. For example, a user could filter by both status, department, and shift to quickly assess the health status of a very specific group of people.

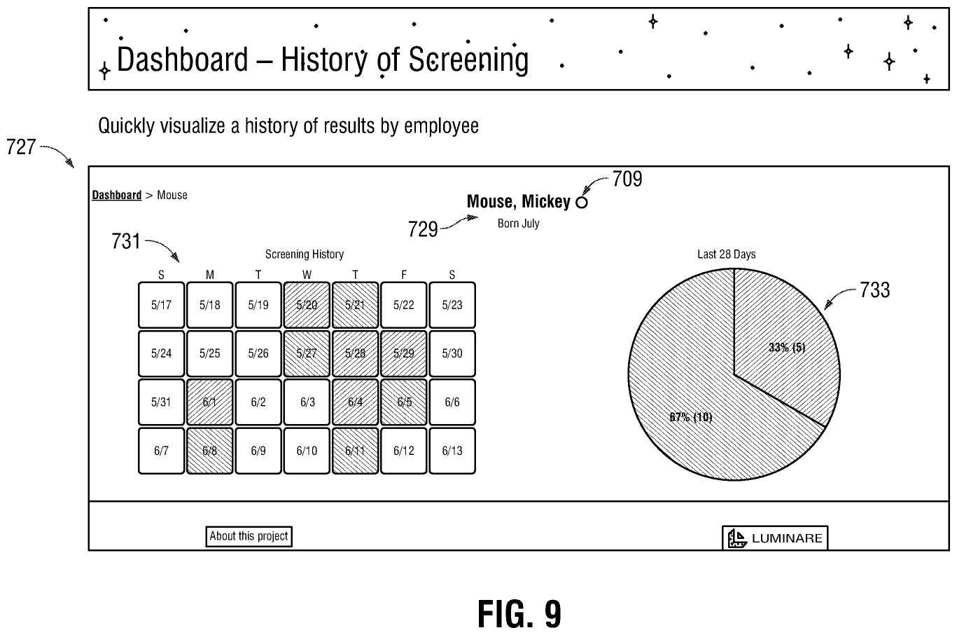

FIG. 9 depicts dashboard 727, presenting the data of a single patient. Dashboard 727 presents a name 709, birthdate 729, screening history calendar 731, and status graph 733. The screening history calendar 731 presents the status of the patient for each day within a timeframe (e.g., a month). The status can be the same as status 703b shown in FIGS. 7 and 8, with red indicate infection or high risk of injection, green indicating no infection or low risk of injection, and gray indicating no data. The status graph 733 can present, by percentage, the status results for the employee over the same timeframe as the screening history calendar 731.

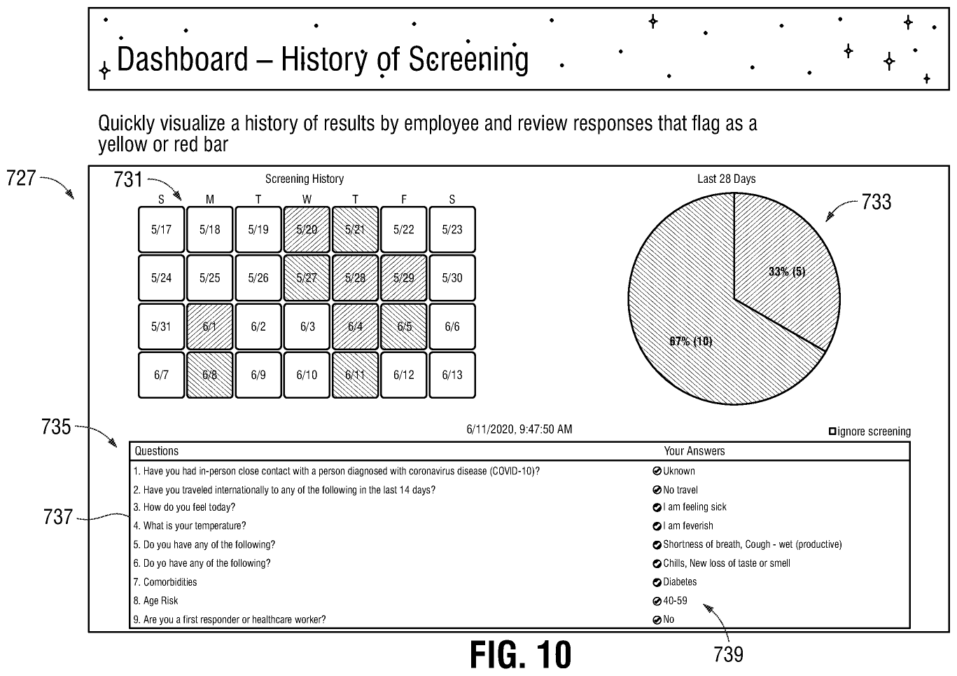

FIG. 10 shows that dashboard 727 can allow a user to review the responses to the questions asked of the employee during the COVID-19 screening (i.e., to view the patient data). Here, the user of the dashboard 727 has selected June 11, as indicated by the outline in the calendar 731. This selection initiates the presentation of the patient data 735 for that day. The patient data 735 includes the questions 737 asked and the answers 739 given in the COVID-19 screening. The answers to each of the questions asked can be indicative of whether or not a person is probable to have COVID-19.

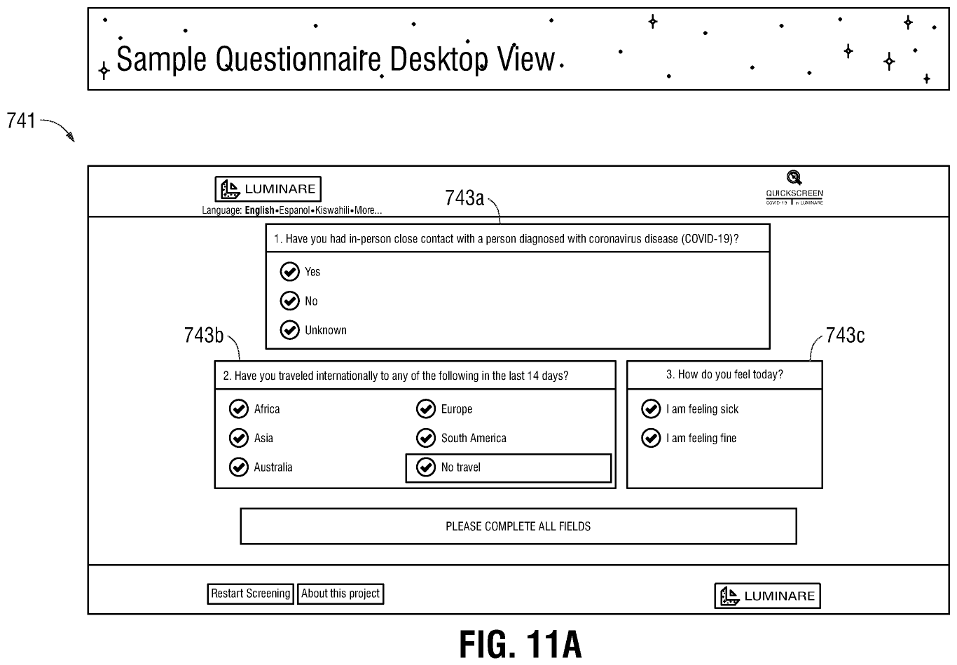

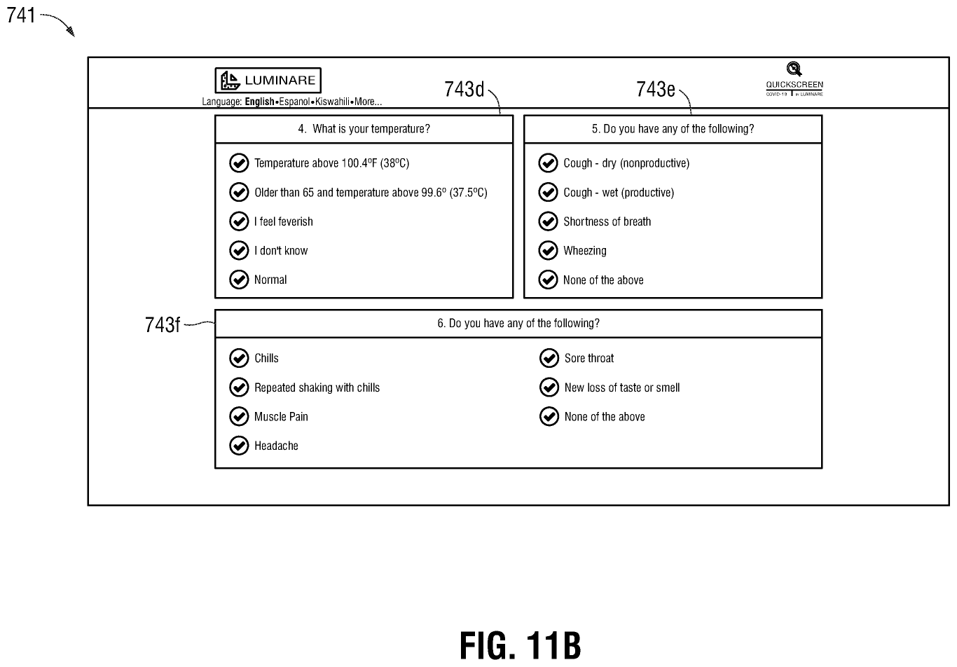

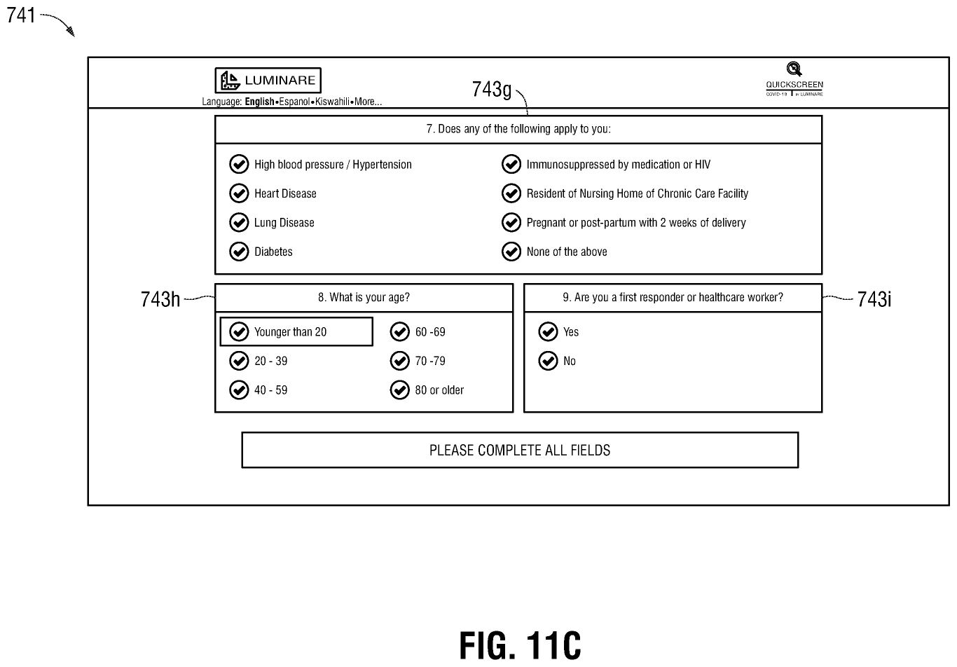

FIGS. 11A-11C depict an exemplary dashboard 741 presenting screening questions 743a-743c for answer by a patient for determining a probability or risk of COVID-19. The exemplary dashboard 741 of FIGS. 11A-11C are presented on a desktop computer.

In addition, box 744 reminds the user to answer all of the diagnostic questions with the message, “PLEASE COMPLETE ALL FIELDS.” This box 744 may change color or the message may change after the user has answered all of the questions. In doing so, the user is able to quickly and easily recognize whether they have answered each and every question. Further, providing such a box encourages a greater number of users to answer every question which, in turn, provides more data for the algorithm to determine the risk of COVID-19.

Further, one or more of the questions and/or list of answers may be hidden from the user until a certain selection is made. For example, the survey may only display yes/no questions at first and only after the user selects yes or no will the dashboard provide an expanded selection of answers. Keeping the questions and answers appear shorter may encourage users to answer truthfully and thoughtfully, thereby increasing the accuracy of the provided data.

As an example, the dashboard may at first ask if the user has any pre-existing health conditions with the provided answers being “Yes” and “No.” If the user selects “Yes”, the dashboard will expand to ask the user another question, such as 743g.

Further, the dashboard may store the answers to certain questions to correct future user errors. For example, if a user inputs that they have a chronic health condition, such as diabetes, the dashboard will store that information for that user so that the user does not have to enter the same information in every time. As another example, a user may have traveled to a high-risk location at a certain date or been in contact with an individual who has tested positive for COVID-19. The dashboard may store the date data of the user and automatically update the answers for the user.

In addition, some answers may automatically cause additional questions to be asked by the dashboard. When a user inputs such answers, it may be useful to ask further questions, such as questions that ask more specific questions to gather more details regarding certain information. For example, if a user inputs that they have traveled to a certain area of concern, such as an area that has recently had a COVID-19 outbreak, the dashboard may ask which cities and which travel hubs were used to enter and exit the area of concern. For example, a user that boarded public transportation may have a higher risk than a user that only used a personal vehicle.

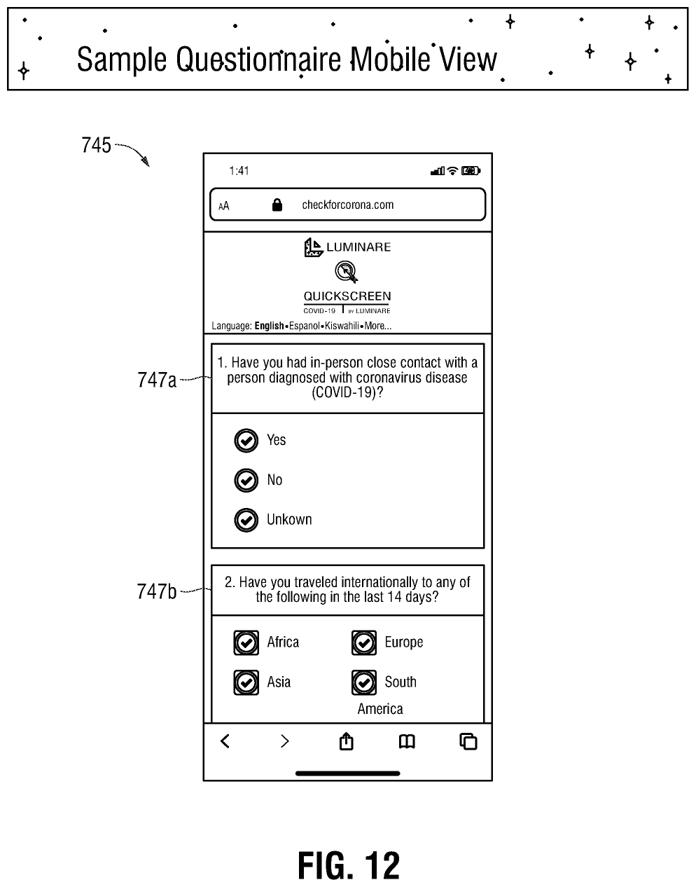

FIG. 12 depicts an exemplary dashboard 745 presenting screening questions 747a-747b for answer by a patient for determining a probability or risk of COVID-19. The exemplary dashboard 745 of FIG. 12 is presented on a mobile device (e.g., an IPHONE®).

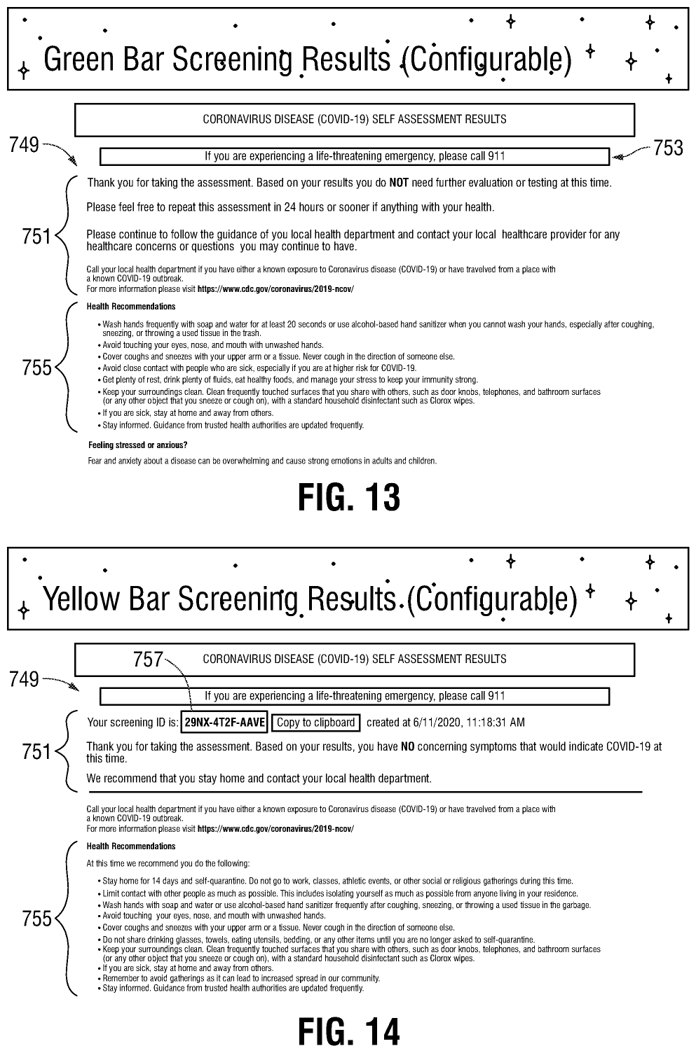

FIG. 13 depicts an exemplary results dashboard 749. In the embodiment shown, the patient is assessed to not need further evaluation or testing, and to not have COVID-19. Results dashboard 749 includes: an indication of results 751, a suggestion to call 911 if needed 753, health recommendations 755.

FIG. 14 depicts an exemplary results dashboard 749. In the embodiment shown, the patient is assessed to no concerning symptoms indicative of COVID-19, but is recommend to stay home and contact a local health department at results 751. Results 751 include an ID 757.

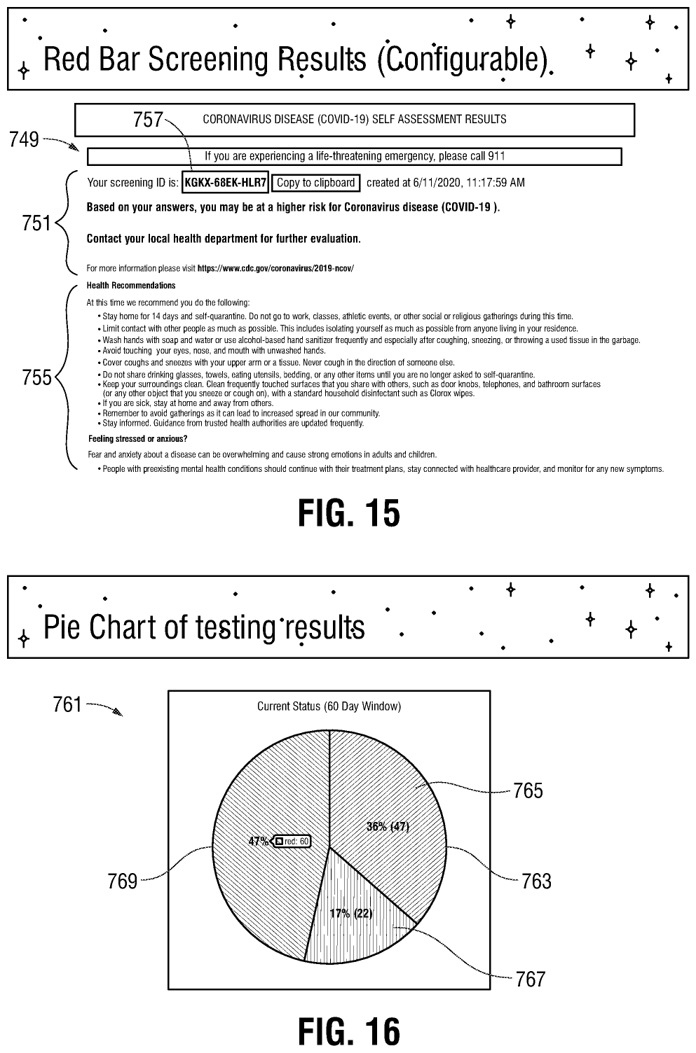

FIG. 15 depicts an exemplary results dashboard 749. In the embodiment shown, the patient is assessed to have a high risk for COVID-19, and is recommend to contact a local health department at results 751.

FIG. 16 depicts status pie chart dashboard 761, including status pie chart 763 having portions that indicate the percentage of no COVID-19 765, low risk of COVID-19 767, and high risk of COVID-19 769. The status pie chart 763 shown is for a 60-day timeframe, but can be for a different timeframe, as desired. As discussed above in reference to FIGS. 13 and 14, a patient may have different results on different days, depending on the answers provided by the patient. The pie chart 763 may show patients that have had at least one or a certain result in a specified time window, or the pie chart 763 can be further refined to show patients that have had multiple of a certain result, such as 2, 3, 4, or more, in a specified time window.

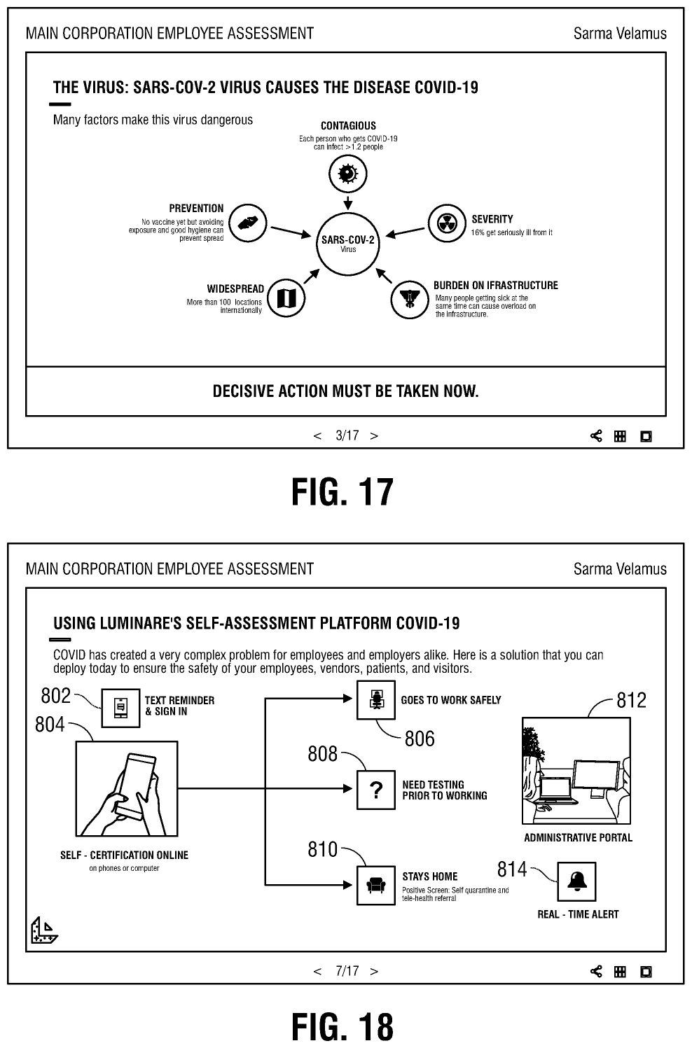

As shown in the chart of FIG. 17, COVID-19 is dangerous and contagious. Without being bound by theory, it is believed that in group settings, such as social gatherings or work environments, are high-risk situations for transmission of the virus. Identifying those at risk (older people, those with underlying medical conditions, etc.), screening for signs and symptoms, requiring those that are sick to stay home, and communicating risk to those in the group are some of the ways that to mitigate the spread of COVID-19. Using the systems, methods, apparatus, and graphical user interfaces disclosed herein, employees can self-certify their condition. For example, as shown in the chart in FIG. 18, an employee can receive a text reminder 802 to sign in to the COVID-19 screening system to answer the screening questions. The employee can answer the questions using a computer or phone, as shown at 804. The screening system can analyze the employee's answers to determine if the employee can: go to work safely, 806; needs testing prior to working, 808; or needs to stay home, self-quarantine, and get a tele-health referral, 810. An administrative portal 812 of the screening system can allow an administrator (e.g., employer) to monitor the answers received and can provide the administrator with real-time alerts 814, such as when an employee is determined to have COVID-19.

In some embodiments, the screening system disclosed herein uses cloud computing and/or cloud storage of data (e.g., Microsoft Azure). In some embodiments, the screening system disclosed herein has data security software architecture therein for protection of privacy. In some embodiments, the screening system disclosed herein uses artificial intelligence and machine learning. The screening system disclosed herein can be used to track both daily symptoms and trailing 14 days symptoms. In some embodiments, the screening system disclosed herein can include a geo-fenced contact tracing feature that allows tracking of the location of “patients” of the system. In some embodiments, the administrator is provided with a real-time analytic dashboard that tracks the status of a workforce and ecosystem. The screening system can be enterprise ready. The screening system can be configured to be HIPAA and ADA compliant by restricting data to only those that need to know it, maintaining employee privacy, masking PHI data, and keeping separate private health data from an employee's file. The screening system can facilitate IRS credit claims by providing documentation of reasons for sick leave. The screening system can facilitate OSHA compliance by allowing investigation of COVID-19 cases, determination of contraction sources, evaluations of work exposure, and documentation of forms (e.g., OSHA Form 300).

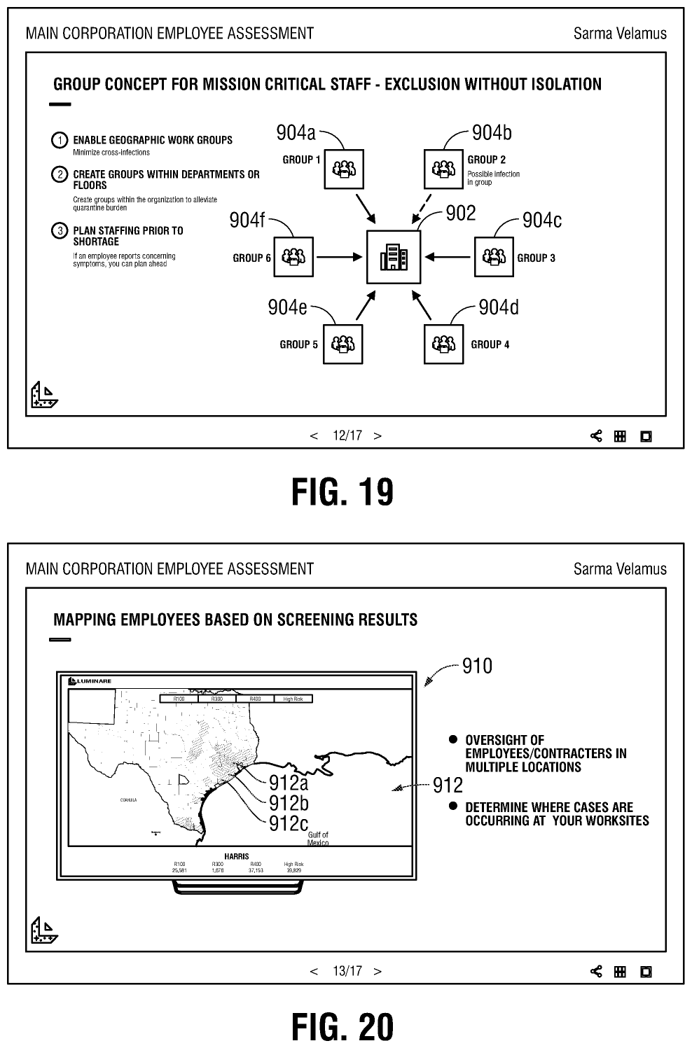

As shown in FIG. 19, the screening system can provide for employee exclusion without isolation. For example, the screening system can be used to: enable geographic workgroups, minimizing cross-infections; create groups within departments or building floors of an organization to alleviate quarantine burden; and/or plant staffing prior to shortages, such as if an employee reports concerning symptoms. In the example in FIG. 19, organization 902 is divided into six groups, 904a-904f. If group 2, 902b, is found to have a possible infection, then that group is already isolated from the other groups 902a and 902c-902f; therefore, the burden of quarantine is only on group 2, 902b.

As shown in FIG. 20, the screening system can provide a dashboard that shows COVID-19 case concentrations in a geographic map format. Dashboard 910 presents map 911, which includes variations in colors 912a-912c that are indicative of variations in the number of or concentration of COVID-19 cases within that region. This allows for oversight of employees and/or contractors in multiple locations, and provides for the determination of where COVID-19 cases are occurring at worksites. While a map of counties in Texas is shown, it should be appreciated that this tracking can be customized to any location or set of locations. For example, an employer with multiple office locations could have a map that shows those different locations and provide a map of a raw number or a percentage of employees having certain results from the above described screening algorithm.

In addition, the tracking tool in FIG. 20 can be used to track the spread of COVID-19 and prevent further spread of COVID-19. For example, if someone who tested positive for COVID-19, or someone who is determined to be at high-risk of having COVID-19 was at one or more locations, this tool can highlight those locations. Further, the tool may provide alerts to others at those locations that they may have been in contact with someone who either tested positive or was at high risk of having COVID-19 so that those individuals may take appropriate precautions. Using such a tool, employers may be able to prevent the spread of COVID-19 through their workforce.

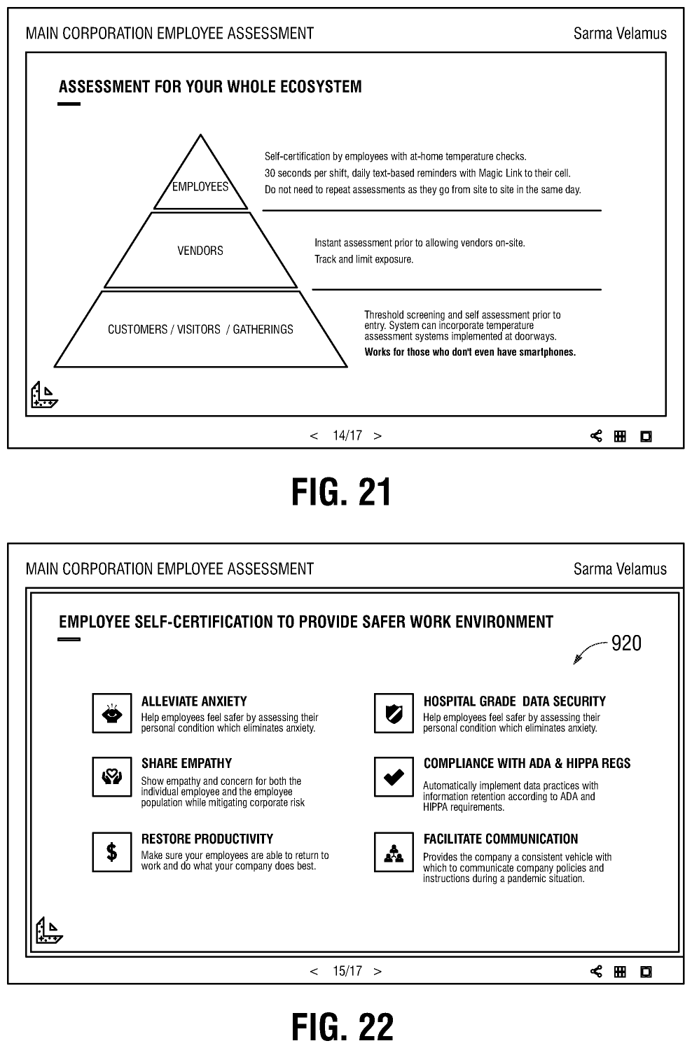

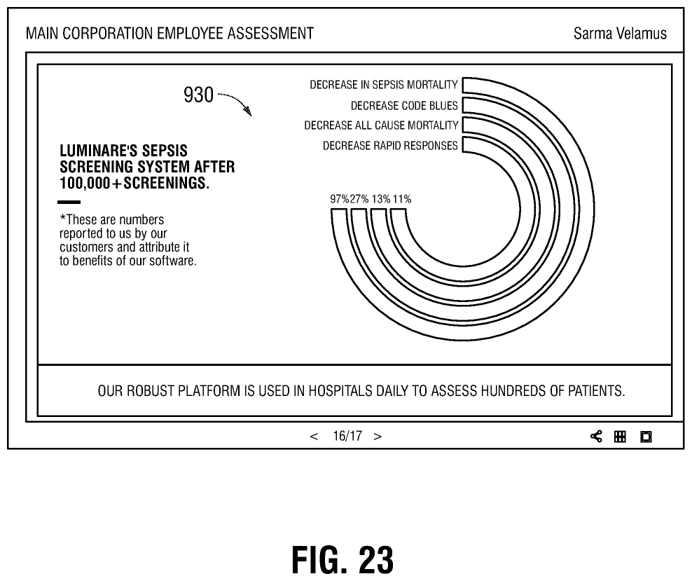

As shown in FIG. 21, the screening system can be used to assess the entire ecosystem of an organization, including employees 914; vendors 916; and customer, visitors, or gatherings. In some embodiments, employees measure their temperature and then enter that temperature into the screening system as part of their “patient data.” In some embodiments, the screening system can be configured to transmit text messages or other messages to employees to remind them to enter data into the system. For vendors, screenings can be performed before the vendors enter the site, and their movements on site can be monitored and controlled. For customer, visitors, or gatherings, screenings can be performed before the vendors enter the site. Temperatures can, in some embodiments, be taken at doorways or other entry points. FIG. 22 provides some of the benefits 920 of having employees self-certify their conditions using the screening system. FIG. 23 depicts some other benefits 930 of using the screening system for other conditions.

The foregoing description has been presented for purposes of illustration and description of preferred embodiments. This description is not intended to limit associated concepts to the various systems, apparatus, structures, and methods specifically described herein. For example, systems and methods described in the context of a patient (potentially) with sepsis, may be applicable to other medical conditions or patients with different and further symptoms (or collections of patient data). As well, systems and components described in the context of the treatment, management or diagnosis of sepsis, may be applicable and beneficial to other medical or patient-physician relationships The embodiments described and illustrated herein are further intended to explain the best and preferred modes for practicing the system and methods, and to enable others skilled in the art to utilize same and other embodiments and with various modifications required by the particular applications or uses.

1. A computer-implemented method of screening a person for infection with coronavirus, said method comprising:
receiving data relating to the person in a computing system, the computing system including a processor and memory coupled to the processor, wherein the memory includes programmable instructions executable by the processor stored thereon, wherein said receiving includes, on a graphical user interface, receiving one or more user selections or inputs; and
determining a risk level of a presence of coronavirus in the person, wherein said determining includes executing at least some of the programmable instructions based on the received data.

2. (canceled)

3. The method of claim 1, wherein the received data comprises an answer to one or more of the following questions:
have you had in-person close contact with a person diagnosed with coronavirus disease;
have you traveled internationally in the last 14 days;
how do you feel today;
what is your temperature;
do you have any symptoms, wherein the symptoms comprise fever, cough, shortness of breath, wheezing, sore throat, loss of taste, loss of smell, muscle pain, headache, shaking, chills, or combinations thereof;
do you have a preexisting condition, wherein the preexisting conditions comprise high blood pressure, heart disease, lung disease, diabetes, immunosuppression, residency at a nursing home or chronic care facility, pregnancy, post-partum, or combinations thereof;
what is your age; and
are you a first responder or healthcare worker.

4. (canceled)

5. (canceled)

6. (canceled)

7. The method of claim 1, where each received datum is linked to a level of risk of having the coronavirus.

8. The method of claim 1, further comprising, after determining the risk level of the presence of coronavirus in the person, presenting the risk level to the person in the graphical user interface.

9. (canceled)

10. The method of claim 1, further comprising presenting health recommendations to the person in the graphical user interface.

11. The method of claim 1, further comprising presenting an administrative dashboard in a graphical user interface of an administrator, wherein the administrative dashboard presents a list of a plurality of people, including an indication of a risk level of a presence of coronavirus in each of the plurality of people.

12. The method of claim 11, wherein the list of the plurality of people is sortable by risk level, department in an organization, shift worked, name.

13. The method of claim 11, wherein the list presents a date screened, a status change, a name, an ID, a location, a department, a shift, a risk level, or combinations thereof for each of the plurality of people on the list.

14. The method of claim 11, wherein, for each person on the list, the graphical user interface of the administrator is configured to present a screening history calendar that presents the risk level for each day within a timeframe, and a status graph that graphically presents a risk level over the timeframe.

15. The method of claim 11, wherein, for each person on the list, the graphical user interface of the administrator is configured to present the received data.

16. The method of claim 9, wherein, if the person is determined to have a level of risk of the presence of coronavirus that is above the threshold level of risk, then the person is assigned a screening ID.

17. The method of claim 11, wherein the graphical user interface of the administrator is configured to present a pie chart of coronavirus testing results.

18. The method of claim 1, further comprising generating a prompt in the graphical user interface to remind the person to enter the data therein.

19. The method of claim 1, wherein, based upon the determined risk level of the presence of coronavirus in the person, the person is advised via the graphical user interface to come to work, obtain a coronavirus test prior to coming to work, or stay at home.

20. The method of claim 11, wherein the administrative dashboard presents the indication of the risk level of the presence of coronavirus in the plurality of people graphically within a map.

21. (canceled)

22. The method of claim 11, further comprising tracking a geographic location of the people having a level of risk of the presence of coronavirus that is above a threshold level of risk.

23. The method of claim 1, wherein said determining includes executing programmable instructions stored in said memory, with said received data indicating, in negative or affirmative, the level of risk of the presence of coronavirus.

24. A computer system for screening a person for infection with coronavirus, said system comprising:
a processor and memory coupled to the processor, wherein the memory includes programmable instructions executable by the processor stored thereon, wherein the programmable instructions comprise:
instructions for generating and presenting a graphical user interface (GUI)
instructions for receiving data in the GUI, the data relating to the person;
instructions for determining a risk level of a presence of coronavirus in the person, wherein said determining is based on the received data.

25. The system of 24, wherein each received datum is linked to a level of risk of having the coronavirus.

26. The system of claim 24, further comprising programmable instructions for presenting the risk level to the person in the graphical user interface.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)
